(19) United States
(12) Patent Application Publication
(10) Pub. No.: US 2025/0248724 A1
(43) Pub. Date: Aug. 7, 2025

(54) Negative Pressure Stent Delivery Systems and Related Methods

(71) Applicant: ASAHI INTECC CO., LTD., Seto-shi (JP)

(72) Inventor: Hirobumi Watanabe, Irvine, CA (US)

(73) Assignee: ASAHI INTECC CO., LTD., Seto-Shi (JP)

(21) Appl. No.: 19/012,455

(22) Filed: Jan. 7, 2025

Related U.S. Application Data
(60) Provisional application No. 63/627,915, filed on Feb. 1, 2024.

(51) Int. Cl. A61B 17/221; A61B 17/00; A61B 17/22

(52) U.S. Cl. CPC A61B 17/221; A61B 17/00234; A61B 2017/00238; A61B 2017/00309; A61B 2017/00477; A61B 2017/00561; A61B 2017/00862; A61B 2017/00867; A61B 2017/00946; A61B 2017/22079; A61B 2017/2215; A61B 2217/005

(57) ABSTRACT

A first guide tube can be advanced through a patient's vasculature, and a covered stent that can be radially expandable from a compressed state to an expanded state and comprise a frame configured to urge the covered stent toward the expanded state when the stent is in the compressed state, a gas-impermeable membrane coupled to the frame, and a lumen surrounded by the gas-impermeable membrane can be advanced through the first guide tube while pressure within the covered stent's lumen is reduced. After advancing the covered stent through the first guide tube, the covered stent can be expanded from the compressed state to the expanded state at least by positioning the covered stent relative to the first guide tube such that at least a portion of the covered stent is disposed distally of the first guide tube's distal end and increasing pressure within the covered stent's lumen.

### CROSS-REFERENCE TO RELATED APPLICATIONS

This application is a non-provisional application that claims priority to U.S. Provisional Patent Application No. 63/627,915, filed on Feb. 1, 2024, the entire contents of which are incorporated into the present application by reference.

### FIELD OF INVENTION

The present invention relates generally to stent delivery systems and methods for removing a thrombus from a patient's vasculature.

### BACKGROUND

A thrombus (also referred to as a blood clot) can block the flow of blood through a vessel, thereby depriving tissues of blood and oxygen and causing damage thereto. Thrombi are the predominant cause of strokes, which require prompt treatment to mitigate the risk of long-term disability and death.

A thrombectomy is a common procedure for treating strokes. In a thrombectomy, a guide catheter is inserted into a patient's vasculature at the groin and advanced therethrough toward the thrombus. A stent retriever can then be passed through the guide catheter and engage the thrombus to capture it; once the thrombus is captured, the stent retriever and catheter can be removed to restore blood flow to the brain. Alternatively, a small-bore aspiration catheter can be passed through the guide catheter and, when its distal end is at the thrombus, a vacuum can be applied at the catheter's proximal end to draw the thrombus against the aspiration catheter's mouth for removal. Over the past decade, thrombectomies have improved the stroke treatment success rate, with about 85% of the procedures achieving recanalization.

However, the inventor has recognized a number of challenges that have prevented successful recanalization in all thrombectomies, or at least increased the difficulty of successful thrombectomies. Some thrombectomies rely on the expansion of a stent in the vasculature for removal of the thrombus. For example, stent retrievers may need to be radially expanded to mechanically engage and accordingly capture the thrombus. And when a thrombectomy relies on aspiration of the thrombus, an expandable, covered stent can be beneficial. This is because aspiration catheters must be able to access the vasculature in which a thrombus is located—commonly the internal carotid artery or middle cerebral artery (e.g., the M1 segment thereof), for strokes—and are accordingly usually relatively narrow, having a diameter that is less than 50% of the diameter of the blood vessel. Such narrow aspiration catheters may not be able to ingest a stroke-inducing thrombus that spans across the blood vessel. As a result, thrombus removal with a narrow aspiration catheter is often achieved by retracting the aspiration catheter with most of the thrombus disposed outside of its lumen, rather than by allowing the vacuum source to draw the thrombus through the lumen. The exposed thrombus is at risk of detachment during catheter withdrawal, which can result in failed recanalization. Some have attempted to use expandable, covered stents to address this challenge, where the stent is advanced through a small-diameter sheath to a thrombus and deployed distally out of the sheath such that a distal portion of the stent expands radially to the artery wall. In this manner, the stent can ingest the thrombus through its expanded mouth. One example of such a device is the Anaconda Biomed S.L. ANCD Advanced Thrombectomy System.

Stent expansion is sometimes achieved using a balloon catheter. With a balloon catheter, the stent typically has a frame comprising stainless steel, is delivered to the target vessel while in a compressed state, and is thereafter expanded using the balloon catheter, which can cause the stent to deform beyond its elastic regime such that the stent plastically and permanently deforms to the expanded state. This relies on the vessel diameter being sufficiently large to allow enough expansion of the stent to achieve permanent deformation; if the vessel is not large enough, the balloon catheter may not successfully cause permanent expansion of the stent, which can be problematic in view of variations in blood vessel diameters.

Self-expanding stents may address some of the challenges of balloon catheters. A self-expanding stent can have a frame comprising a superelastic, shape-memory alloy such as nitinol that can urge the stent to its expanded state when the stent is in the compressed state. To deliver the self-expanding stent to the thrombus, the stent can be compressed and advanced in the vasculature through a sheath such that when the self-expanding stent is deployed out of the sheath, it self-expands to the vessel wall with enough force to create a seal such that the thrombus can be aspirated into the stent. While this self-expansion can allow the stent to be used in differently-sized vessels, the radially-outward urging of the stent can promote friction between the stent and the sheath through which it is advanced, which can in turn impede the stent's advancement through the vasculature. Cooling the self-expanding stent with, for example, liquid nitrogen before advancing it through the vasculature can decrease the radially-outward urging forces generated by the stent's frame and can thus mitigate the friction resulting therefrom; when the stent is advanced through the vasculature, it can be heated due to the body's higher temperature, which—due to the shape-memory alloy of the stent's frame—can increase the force with which the stent's frame urges the stent radially outward to its expanded state. The temperature of the stent may increase relatively fast and may reach body temperature before the self-expanding stent reaches the most tortuous part of the vasculature such that, even with the pre-advancement cooling, friction can still limit the stent's advancement to the thrombus. As a result, a self-expanding, covered stent in current systems usually does not directly capture the thrombus through aspiration; instead, the self-expanding, covered stent typically serves as a protection device, where another mechanism such as a stent retriever is advanced past the self-expanding stent, captures a thrombus, and retracts the thrombus to the self-expanding stent for removal.

### SUMMARY

To address these challenges, some of the present stent delivery systems can be configured to reduce pressure within the lumen of a covered stent that has a gas-impermeable membrane and a frame configured to urge the covered stent to an expanded state when the covered stent is in a compressed state. The pressure within the covered stent's lumen can be reduced before the covered stent is advanced through the vasculature of a patient. The pressure reduction can yield a pressure differential across the covered stent's membrane that urges the covered stent radially inward toward the compressed state and can counteract the radially-outward forces generated by the covered stent's frame. That can in turn reduce the friction between the covered stent and the surface(s) (e.g., of one or more tubes) that the stent passes over as it advances through the patient's vasculature when in the compressed state, which can accordingly facilitate the stent's advancement to the thrombus. The covered stent can thus more-readily reach the thrombus, where the covered stent can be deployed at least by positioning at least a portion of the covered stent distally of any tube(s) through which it was advanced and increasing pressure within the covered stent's lumen such that the frame can urge the covered stent radially outward to the expanded state to ingest the thrombus.

Pressure within the covered stent's lumen can be reduced using, for example, a delivery assistance tube having a distal portion that can be configured to be positioned within the covered stent's lumen when the covered stent is in the compressed state. The delivery assistance tube's distal portion can comprise a plurality of openings extending through an outer wall of the delivery assistance tube, and the delivery assistance tube can include a lumen that extends from a proximal end of the delivery assistance tube to the distal portion of the delivery assistance tube. As a result, reducing pressure at the proximal end of the delivery assistance tube can cause gas to flow from the delivery assistance tube's distal portion to its proximal end through its lumen and accordingly reduce pressure within the distal portion. When the distal portion is disposed in the covered stent's lumen, that pressure reduction can be communicated to the covered stent's lumen via the distal portion's openings such that the covered stent is drawn radially inward around the distal portion. The covered stent's gas-impermeable membrane can seal the openings of the delivery assistance tube's distal portion to maintain the pressure reduction as the covered stent is advanced through the patient's vasculature.

In other embodiments, pressure within the covered stent's lumen can be reduced using a pusher and a compliant bead that is coupled to the pusher. The compliant bead can be positionable within the covered stent's lumen and can create a seal with an inner surface of the covered stent in a distal portion thereof when the covered stent is in the compressed state such that gas cannot flow proximally past the compliant bead in the covered stent's lumen. With such a seal, pressure can be reduced proximally of the covered stent—such as at a proximal end of an aspiration tube that is coupled to the covered stent's proximal end—to evacuate gas from the covered stent's lumen and accordingly reduce the pressure therein, thereby urging the covered stent radially inward toward the compressed state to facilitate the covered stent's advancement through the patient's vasculature.

Some details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1A is a top view of a first embodiment of the present stent delivery systems that includes a stent assembly comprising a covered stent, a delivery assistance tube, and a first guide tube.

FIG. 1B is a sectional view of the stent assembly of the system of FIG. 1A taken along line 1B-1B of FIG. 1A when the covered stent is in the expanded state, where the proximal end of the covered stent is coupled to an aspiration tube.

FIG. 1C is a sectional view of the delivery assistance tube of the system of FIG. 1A taken along line 1C-1C of FIG. 1A and illustrates how a lumen of the delivery assistance tube is in fluid communication with openings that extend through an outer wall of the delivery assistance tube in a distal portion of the delivery assistance tube.

FIG. 1D is a sectional view of the first guide tube of the system of FIG. 1A taken along line 1D-1D of FIG. 1A.

FIG. 1E is a top view of the system of FIG. 1A in an insertion configuration where the covered stent of the stent assembly is disposed within a lumen of the first guide tube and the distal portion of the delivery assistance tube is positioned within a lumen of the covered stent.

FIG. 1F is a sectional view of the system of FIG. 1A taken along line 1F-1F of FIG. 1E.

FIG. 1G is an enlarged sectional view of a distal portion of the system of FIG. 1A while the system is in the insertion configuration where the covered stent of the stent assembly is disposed within a lumen of the first guide tube and the distal portion of the delivery assistance tube is disposed within the lumen of the covered stent.

FIG. 2 is a sectional view of the stent assembly of FIG. 1B when the covered stent is in the compressed state.

FIG. 3A is a sectional view of a stent assembly of some of the present stent delivery systems that is substantially the same as the stent assembly of FIG. 1B, except that the FIG. 3A assembly does not include an aspiration tube coupled to the proximal end of the covered stent and the FIG. 3A assembly includes a seal disposed around a proximal portion of the FIG. 3A covered stent. The covered stent in FIG. 3A is in the expanded state.

FIG. 3B is a sectional view of the stent assembly of FIG. 3A while the covered stent is in the compressed state.

FIG. 3C is a sectional view of a second embodiment of the present stent delivery systems that is substantially the same as the stent delivery system of FIG. 1A, except that the FIG. 3C system uses the stent assembly of FIG. 3A rather than the stent assembly of FIG. 1B. FIG. 3C shows its system in an insertion configuration where the system's covered stent is disposed within a lumen of the first guide tube and the distal portion of the delivery assistance tube is positioned within a lumen of the covered stent.

FIG. 4 is a sectional view of a delivery assistance tube of some of the present stent delivery systems that is substantially the same as the delivery assistance tube of FIG. 1B except that the delivery assistance tube of FIG. 4 includes two lumens, where a first one of the lumens that extends to the distal portion of the delivery assistance tube and is in fluid communication with the openings of the delivery assistance tube surrounds a second lumen that extends from the proximal end of the delivery assistance tube to a distal end of the delivery assistance tube such that the distal end of the delivery assistance tube includes an opening.

FIGS. 5A and 5B are enlarged top and side views, respectively, of the distal portion of the delivery assistance tube of FIG. 1B and illustrates how the openings thereof includes a plurality of first pairs of slits and a plurality of second pairs of slits.

FIG. 5C is a sectional view of the delivery assistance tube of FIG. 1B taken along line 5C-5C of FIG. 5A and illustrates how the outer wall of the delivery assistance tube defines one of the first pairs of slits.

FIG. 5D is a sectional view of the delivery assistance tube of FIG. 1B taken along line 5D-5D of FIG. 5A and illustrates how the outer wall of the delivery assistance tube defines one of the second pairs of slits.

FIGS. 6A and 6B illustrate the system of FIG. 1A in the insertion configuration and while the proximal end of the delivery assistance tube is coupled, via a valve, to a negative pressure source and a positive pressure source. In FIG. 6A, the valve is in a first state in which the negative pressure source is in fluid communication with the lumen of the delivery assistance tube, and in FIG. 6B the valve is in a second state in which the positive pressure source is in fluid communication with the lumen of the delivery assistance tube.

FIGS. 7A and 7B illustrate insertion of the covered stent of the system of FIG. 1A into the lumen of the system's first guide tube while the covered stent is disposed in the lumen of an introducer tube.

FIG. 7C illustrates the covered stent of the system of FIG. 1A disposed in a proximal portion of the lumen of the system's first guide tube after the introducer tube is removed from the first guide tube's lumen.

FIG. 7D is an enlarged sectional view of the distal portion of the system of FIG. 1A and illustrates advancement of the covered stent of the system to the distal end of the system's first guide tube while the distal portion of the system's delivery assistance tube is disposed in the lumen of the covered stent.

FIG. 7E is an enlarged sectional view of the distal portion of the system of FIG. 1A while the system is an a deployment configuration where the covered stent is positioned relative to the first guide tube such that at least a portion of the covered stent is disposed distally of the distal end of the first guide tube and the distal portion of the delivery assistance tube is disposed within the lumen of the covered stent. In FIG. 7E, via the delivery assistance tube, pressure is reduced within the lumen of the covered stent such that the covered stent is in the compressed state.

FIG. 7F is an enlarged sectional view of the distal portion of the system of FIG. 1A while the system is in the deployment configuration, where pressure is no longer reduced within the lumen of the covered stent such that the covered stent is in the expanded state.

FIG. 7G is an enlarged sectional view of the distal portion of the system of FIG. 1A while the system is in an aspiration configuration in which the covered stent is in the expanded state and the delivery assistance tube is removed from the lumen of the covered stent.

FIG. 8 is an enlarged sectional view of the distal portion of a third embodiment of the present stent delivery systems that is substantially the same as the system of FIG. 1A except that the FIG. 8 system does not include a delivery assistance tube and instead comprises a pusher configured to be received in the lumen of the covered stent and a compliant bead that is coupled to the pusher and configured to sealingly engage a distal portion of the covered stent when the covered stent is in the compressed state such that gas is not permitted to flow from a distal end of the covered stent to a portion of the lumen of the covered stent that is proximal of the compliant bead.

FIG. 9 is an enlarged sectional view of the distal portion of a fourth embodiment of the present stent delivery systems that is substantially the same as the system of FIG. 8 except that there is a lumen extending through the pusher and the compliant bead of the system of FIG. 9.

FIG. 10A depicts vasculature of a patient with a thrombus positioned in the M1 segment of the middle cerebral artery.

FIG. 10B depicts the vasculature of FIG. 10A, with the first guide tube of the FIG. 1A system extending through and past a second guide tube to the middle cerebral artery. In FIG. 10B, a distal end of the second guide tube is positioned proximally of the internal carotid artery.

FIG. 10C is an enlarged sectional view of the distal portion of the FIG. 1A system disposed in the FIG. 10A vasculature, where the system is in the insertion configuration such that the distal ends of the first guide tube, covered stent, and delivery assistance tube are positioned at the thrombus.

FIG. 10D is an enlarged sectional view of the distal portion of the FIG. 1A system disposed in the FIG. 10A vasculature, where the system is in the deployment configuration and pressure within the lumen of the covered stent is reduced such that the covered stent is in the compressed state.

FIG. 10E is an enlarged sectional view of the distal portion of the FIG. 1A system disposed in the FIG. 10A vasculature, where the system is in the deployment configuration and pressure within the lumen of the covered stent is no longer reduced such that the covered stent is in the expanded state.

FIG. 10F is an enlarged sectional view of the distal portion of the FIG. 1A system disposed in the FIG. 10A vasculature, where the system is in the aspiration configuration in which the delivery assistance tube is removed from the lumen of the covered stent.

FIGS. 10G-10J are enlarged sectional views of the distal portion of the FIG. 1A system disposed in the FIG. 10A vasculature while the system is in the aspiration configuration and illustrate aspiration of the thrombus through the covered stent for removal.

### DETAILED DESCRIPTION

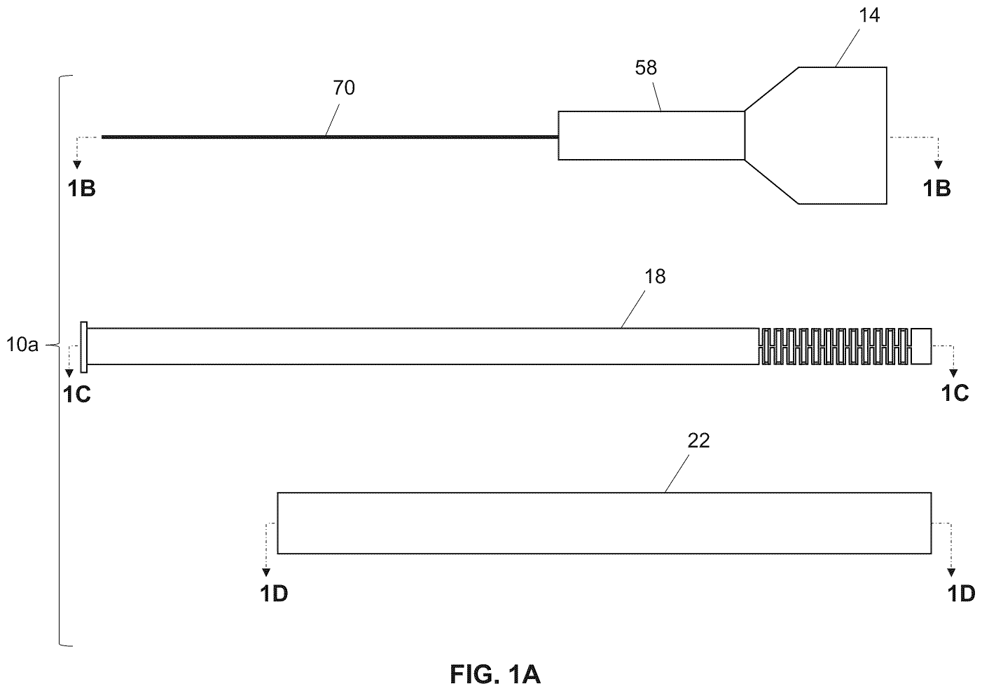

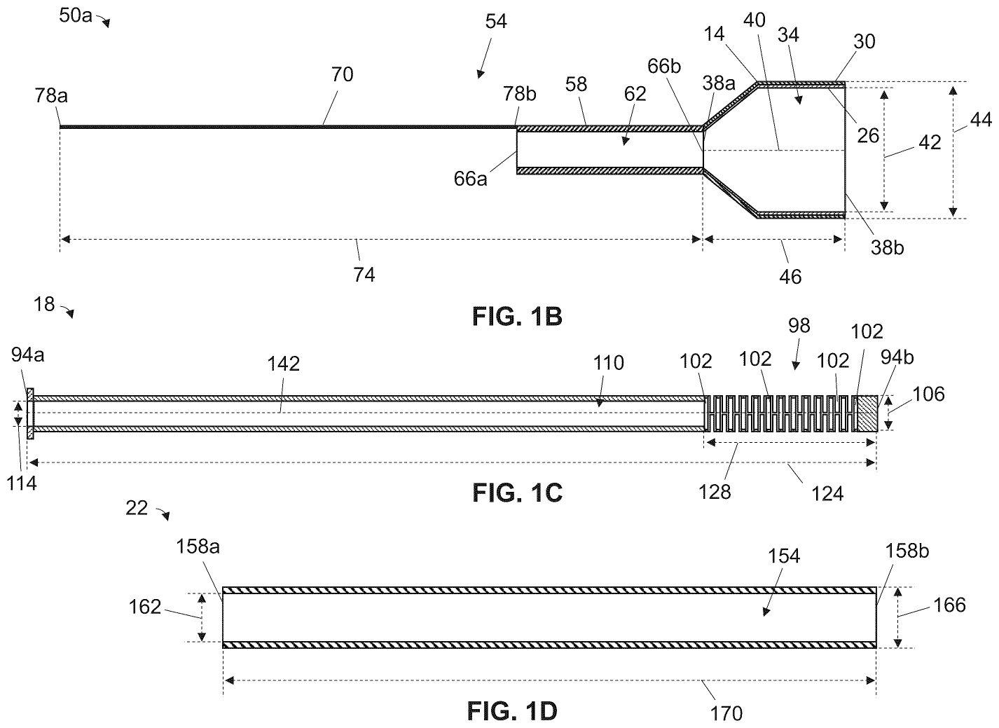

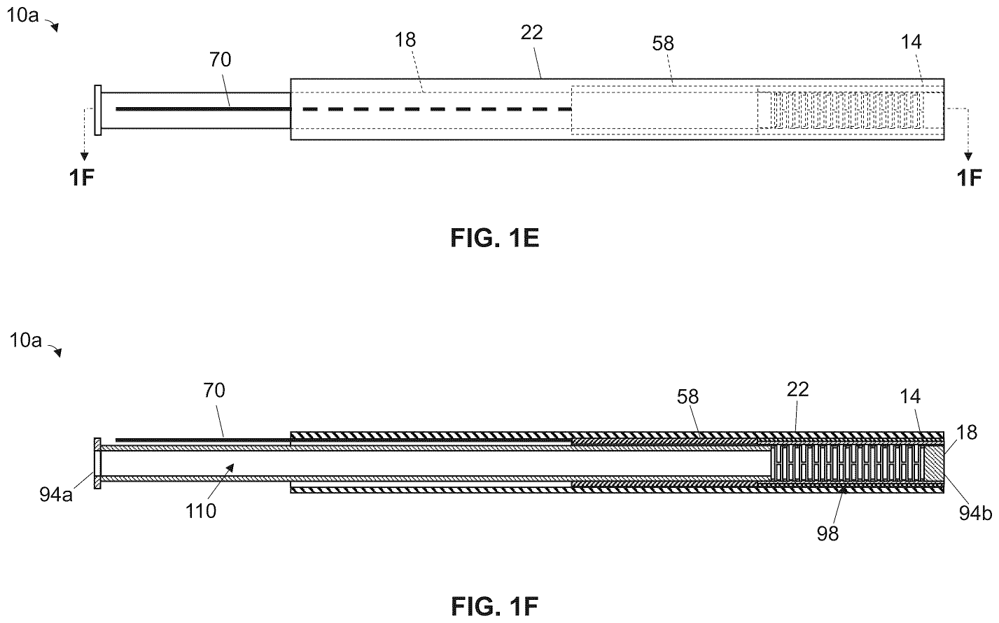

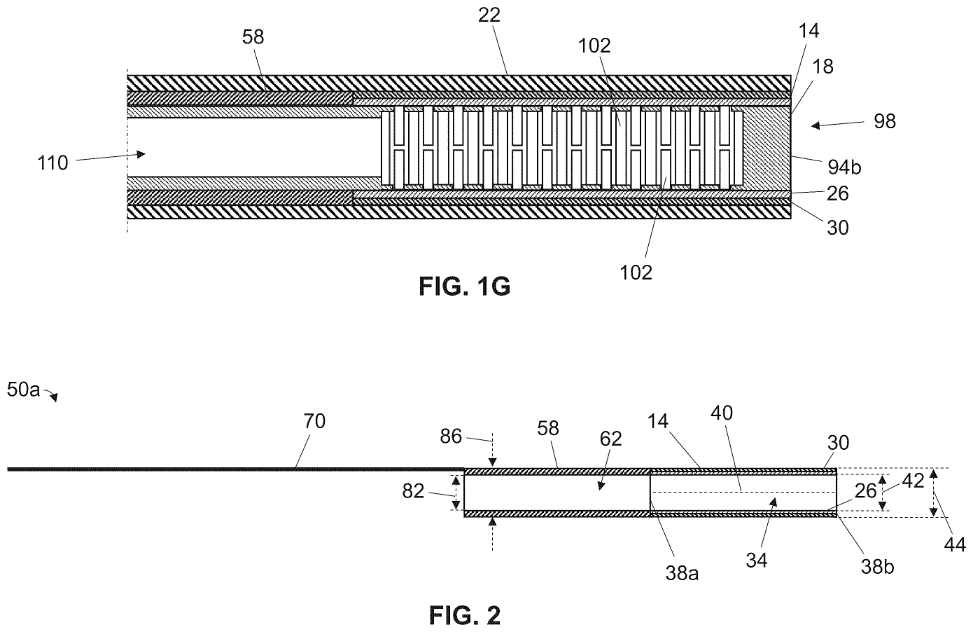

Referring to FIGS. 1A-1G, shown in a first embodiment 10a of the present stent delivery systems that can comprise a covered stent 14 and one or more components to facilitate delivery of the covered stent to a thrombus in the vasculature of a patient, such as a delivery assistance tube 18 and/or a first guide tube 22.

Referring specifically to FIG. 1B and additionally to FIG. 2, covered stent 14 can be radially expandable to an expanded state (FIG. 1B) from a compressed state (FIG. 2), where a maximum internal transverse dimension 42 (e.g., diameter) and a maximum external transverse dimension 44 (e.g., diameter) of the covered stent (e.g., at a distal end 38b of the covered stent), measured perpendicularly to a longitudinal axis 40 that extends between proximal and distal ends 38a and 38b of the covered stent, are each larger when the covered stent is in the expanded state than when the covered stent is in the compressed state. As an illustration, covered stent 14's internal transverse dimension 42 and/or external transverse dimension 44 when the covered stent is in the compressed state can be less than or equal to any one of, or between any two of, 80%, 70%, 60%, 50%, 40%, or 30% of the internal transverse dimension and/or external transverse dimension, respectively, of the covered stent when the covered stent is in the expanded state. Covered stent 14's expandability can allow the covered stent to be in the compressed state when it is delivered to a thrombus to facilitate its advancement through a patient's vasculature, and to thereafter be deployed in the expanded state for removal of the thrombus through the covered stent as described in further detail below.

When in the expanded state (FIG. 1B), covered stent 14 can be sized to contact the vessel walls in the patient's vasculature and to ingest the thrombus such that the thrombus can enter the covered stent through its distal end 38b and pass through the covered stent's lumen 34 that extends between the covered stent's proximal and distal ends 38a and 38b. For example, for use in a patient's neurovasculature, when fully expanded covered stent 14's maximum internal transverse dimension 42 can be greater than or equal to any one of, or between any two of, 0.25, 0.31, 0.37, 0.43, 0.49, 0.55, or 0.61 centimeters (cm) (e.g., at least 0.31 cm) and its external transverse dimension 44 can be greater than or equal to any one of, or between any two of, 0.38, 0.44, 0.50, 0.56, 0.62, 0.68, or 0.74 cm (e.g., at least 0.4445 cm). Furthermore, covered stent 14 can comprise a membrane 30 that can surround the covered stent's lumen 34 such that the membrane can occlude flow between portions of a patient's vasculature that are proximal and distal of covered stent 14 when the covered stent is in the expanded state in the vasculature, and the membrane can accordingly facilitate the drawing of a thrombus into the covered stent's lumen. Suitable materials for membrane 30 include polymers such as polytetrafluoroethylene (PTFE) and urethane; PTFE, for example, advantageously exhibits low friction with other surfaces and thus facilitates insertion and deployment of covered stent 14.

When in the compressed state (FIG. 2), covered stent 14 can be received in a lumen 154 of system 10a's first guide tube 22 that extends between the first guide tube's proximal and distal ends 158a and 158b (FIGS. 1D-1G), and the first guide tube can be sized to be advanced through a patient's vasculature such that the covered stent can advance toward a thrombus through the first guide tube, which can facilitate advancement of the covered stent. For example, an internal transverse dimension 162 (e.g., diameter) of first guide tube 22 can be greater than or equal to any one of, or between any two of, 0.10, 0.11, 0.12, 0.13, 0.14, 0.15, 0.16, 0.17, 0.18, 0.19, 0.20, 0.21, 0.22, or 0.23 cm (e.g., greater than or equal to 0.13 cm) and an external transverse dimension 166 (e.g., diameter) of the first guide tube can be less than or equal to any one of, or between any two of, 0.24, 0.23, 0.22, 0.21, 0.20, 0.19, 0.18, 0.17, or 0.16 cm (e.g., less than or equal to 0.22 cm) (e.g., the first guide tube can be a 6 F catheter).

The components of system 10a can each have a length to permit a user to advance covered stent 14 to a thrombus. For example, length 170 of first guide tube 22 (FIG. 1D), measured between its proximal and distal ends 158a and 158b, can be sufficient to allow the first guide tube to be advanced from an insertion point at a patient's groin to the internal carotid artery (ICA) or middle cerebral artery (MCA) (e.g., the M1 segment thereof), such as greater than or equal to any one of, or between any one of, 80, 90, 100, 110, 120, 130, or 140 cm (e.g., at least 90 cm). Covered stent 14 can thus be advanced from the groin to the ICA or MCA through first guide tube 22. Furthermore, covered stent 14 can have a length 46, measured between its proximal and distal ends 38a and 38b, that is adequate to capture and ingest a thrombus (e.g., after retraction of first guide tube 22, as explained in further detail below). For example, covered stent 14's length 46 (e.g., when the covered stent is in the expanded state) can be greater than or equal to any one of, or between any two of, 1.5, 2, 2.5, 3, 3.5, 4, 4.5, 5, 6, 7, 8, 9, 10, 11, 12, 13, or 14 cm. Additionally, covered stent 14 can be part of a stent assembly 50a having one or more extension components 54 coupled to and disposed proximally of the covered stent, where a collective length 74 of the extension component(s) allows the covered stent to reach, via manipulation of the extension component(s), the thrombus from a point at which the covered stent enters the patient's vasculature. For example, extension component(s) 54 can include an aspiration tube 58 and/or a pusher 70, and a length 74 of the extension component(s) (e.g., of the aspiration tube if the only extension component, of the pusher if the only extension component, or the combined length of the aspiration tube and length if both are used) can be greater than or equal to any one of, or between any one of, 90, 100, 110, 120, 130, 140 or 150 cm (e.g., at least 110 cm), which can allow, for example, covered stent 14 to be advanced to the ICA or MCA by pushing the extension component(s). A most-proximal one of such extension component(s) 54 can remain accessible outside of the patient's vasculature (e.g., with the proximal end of the most-proximal extension component disposed proximally of first guide tube 22's proximal end 158a (FIGS. 1E and 1F)) when covered stent 14 is at the ICA or MCA such that a user can advance or withdraw the covered stent by pushing or pulling, respectively, the most-proximal extension component.

As shown, extension component(s) 54 can include an aspiration tube 58 having a lumen 62 extending between proximal and distal ends 66a and 66b of the aspiration tube, where the aspiration tube's distal end can be coupled to covered stent 14's proximal end 38a such that a thrombus ingested through the covered stent's lumen 34 can subsequently pass through the aspiration tube's lumen. An internal transverse dimension 82 (e.g., diameter) of aspiration tube 58 can be sufficiently large to facilitate such ingestion and the aspiration tube's external transverse dimension 86 (e.g., diameter) can be sufficiently small to facilitate the passage of stent assembly 50a through a patient's tortuous vasculature and to pass through optional first guide tube 22. For example, aspiration tube 58's internal transverse dimension 82 can be greater than or equal to any one of, or between any two of, 0.05, 0.06, 0.07, 0.08, 0.09, 0.10, 0.11, 0.12, 0.13, 0.14, 0.15, or 0.16 cm (e.g., greater than or equal to 0.09 cm), and its external transverse dimension 86 can be less than or equal to any one of, or between any two of, 0.24, 0.23, 0.22, 0.21, 0.20, 0.19, 0.18, 0.16, 0.15, 0.14, or 0.13 cm (e.g., less than or equal to 0.16 cm, such as about 0.14 cm) (e.g., the aspiration tube can be a 4.2 F catheter). Stent assembly 50a can also comprise a pusher 70 (e.g., a rod or guide wire) extending between proximal and distal ends 78a and 78b, where the distal end of the pusher is coupled to a proximal end of aspiration tube 58 such that pushing or pulling the pusher causes the aspiration tube and covered stent 14, when in the patient's vasculature, to advance toward or withdraw away from the thrombus. Pusher 70 can thus permit a user to move stent assembly 50a even when aspiration tube 58 extends along only a portion of the path between the insertion point and covered stent 14 in the patient's vasculature and is thus not accessible outside of the vasculature. With such a configuration, the smaller-transverse-dimension and thus more-flexible aspiration tube 58 can be more-readily advanced deeper into the patient's vasculature than a larger-transverse-dimension and thus less-flexible tube (such as first guide tube 22) for aspiration of a thrombus proximally through the aspiration tube's lumen 62, and the thrombus can then continue proximally through one or more larger-transverse-dimension tubes (e.g., at least the first guide tube) through which the thrombus can more-readily pass (e.g., due to their larger transverse dimension).

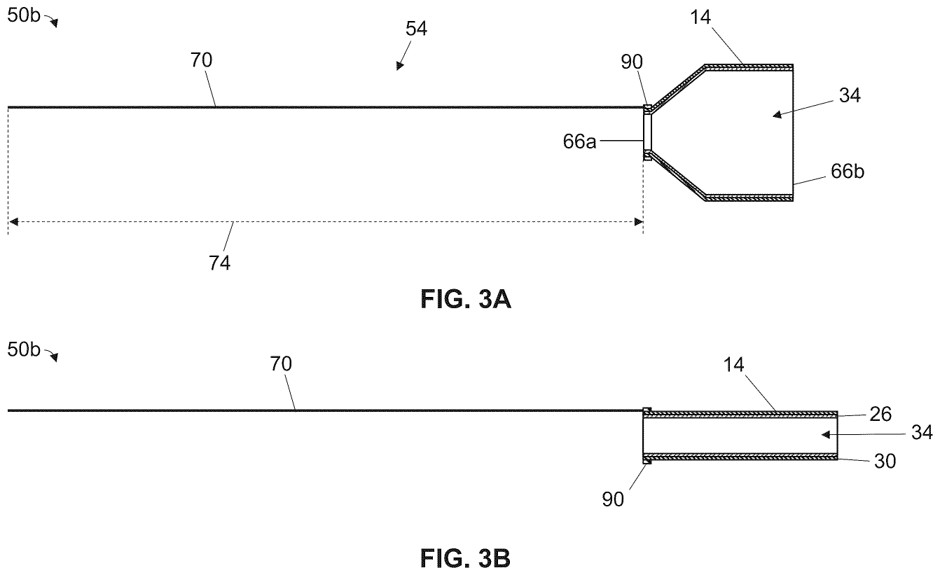

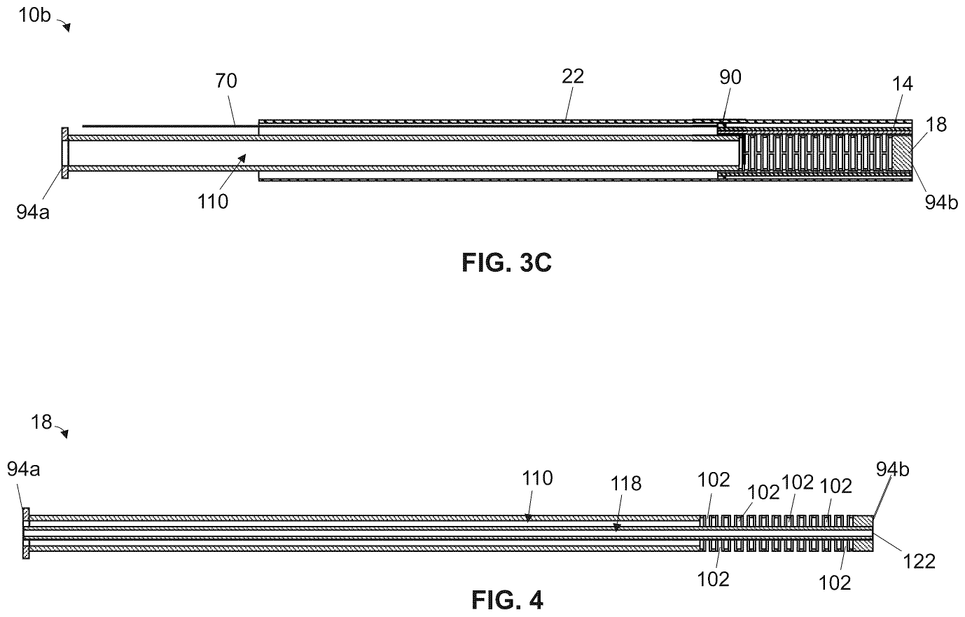

In other embodiments, extension component(s) 54 need not include a pusher 70, such as with aspiration tube 58 being the only extension component and having sufficient length to allow access to the aspiration tube outside of the vasculature when covered stent 14 is at a target location in the vasculature (e.g., the ICA or MCA). Furthermore, and referring to FIGS. 3A and 3B—which show a second embodiment 50b of the present stent assemblies-extension component(s) 54 need not include an aspiration tube 58 and can include, for example, only pusher 70. In some of such embodiments, stent assembly 50b can comprise a seal 90 disposed around a proximal portion of covered stent 44 and coupled to pusher 70, where as shown in FIG. 3C the seal can sealingly engage an inner wall of guide tube 22 when covered stent 14 is disposed therein. In this way, a thrombus ingested through lumen 34 of covered stent 14 can pass directly into guide tube 22's lumen 154 whose larger-transverse-dimension can better facilitate aspiration of the thrombus for removal.

To allow covered stent 14 to expand from the compressed state to the expanded state after being advanced through the patient's vasculature for ingestion of the thrombus, the covered stent can have a frame 26 that is configured to urge the covered stent toward the expanded state when the stent is in the compressed state. For example, frame 26 can comprise a braid and/or struts and can comprise a material that allows the frame to regain its original, expanded shape when covered stent 14 is in the compressed state. Such a material can comprise, to illustrate, nitinol (i.e., an alloy comprising nickel and titanium) that is superelastic and configured to expand when heated and/or stainless steel. Frame 26 can be configured to urge covered stent 14 toward the expanded state with adequate force to form a seal with a vessel wall; for example, when the covered stent is in the compressed state and a surface surrounds the covered stent, a pressure that the covered stent exerts on the surface can be greater than or equal to any one of, or between any two of, 60, 65, 70, 75, 80, 85, 90, 95, or 100 kilopascals (kPa) (e.g., at least 85 kPa). Frame 26 can be coupled to membrane 30 in any suitable manner, such as with the frame adhered to an inner surface of the membrane or embedded within the membrane.

Frame 26's urging of covered stent 14 to the expanded state when the covered stent is in the compressed state can, if not countered, promote friction between the covered stent and the surface(s) that the covered stent contacts while being advanced in a patient's vasculature toward a thrombus (e.g., with an inner surface of first guide tube 22), which can in turn impede such advancement. To counter that urging during advancement of covered stent 14, system 10a can be configured to reduce pressure within the covered stent's lumen 34 to yield a pressure differential between the lumen and an environment outside of the lumen (e.g., across membrane 30) that generates a radially-inward force that counters the radially-outward urging force generated by frame 26.

Referring specifically to FIG. 1C, such a pressure reduction can be achieved via at least system 10a's delivery assistance tube 18. As shown, delivery assistance tube 18 can extend between proximal and distal ends 94a and 94b and can comprise a distal portion 98 that includes the distal end and has a plurality of openings 102 that extend through an outer wall of the delivery assistance tube, such as greater than or equal to any one of, or between any two of, 25, 50, 75, 100, 125, 150, 175, 200, 225, 250, 275, or 300 openings (e.g., at least 200 openings). As shown in FIGS. 1F and 1G, delivery assistance tube 18's distal portion 98 can be configured to be positioned within lumen 34 of covered stent 14 (e.g., while the covered stent is in the compressed state). For example, a maximum transverse dimension 106 (e.g., diameter) of delivery assistance tube 18's distal portion 98 can be less than or equal to any one of, or between any two of, 0.20, 0.19, 0.18, 0.17, 0.16, 0.15, 0.14, 0.13, 0.12, 0.11, or 0.10 cm (e.g., less than or equal to 0.16 cm) and a length 128 of the delivery assistance tube's distal portion can be approximately the same as length 46 of covered stent 14 when the covered stent is in the compressed state, such as greater than or equal to any one of, or between any two of, 1.75, 2.25, 2.75, 3.25, 3.75, 4.25, 4.75, 5.25, 6, 7, 8, 9, 10, 11, 12, 13, 14, or 15 cm and/or less than or equal to any one of, or between any two of, 15%, 14%, 13%, 12%, 11%, 10%, 9%, 8%, 7%, 6%, or 5% of the delivery assistance tube's length 124. Delivery assistance tube can comprise a lumen 110 that extends from the delivery assistance tube's proximal end 94a to the delivery assistance tube's distal portion such that reducing pressure at the proximal end of the delivery assistance tube causes gas in the distal portion to evacuate toward the delivery assistance tube's proximal end through the lumen. When delivery assistance tube 18's distal portion 98 is disposed in lumen 34 of covered stent 14 and the delivery assistance tube is compressed around the distal portion, that gas evacuation can cause a pressure reduction in distal portion 98 that—via openings 102 that can be in fluid communication with lumen 110 through which gas is evacuated—is communicated to the covered stent's lumen to yield a pressure differential that urges the covered stent radially inward around the delivery assistance tube's distal portion. Covered stent 14's membrane 30, because it can be gas impermeable, can seal openings 102 to prevent the ingress of gas outside of lumen 34 and accordingly maintain the pressure reduction caused by the gas evacuation through delivery assistance tube 18's lumen 110. Furthermore, distal end 94b of delivery assistance tube 18 can be closed such that there are no gas ingress points into distal portion 98 and thus covered stent 14's lumen 34 when pressure is reduced at the delivery assistance tube's proximal end 94a. Alternatively, and referring additionally to FIG. 4, delivery assistance tube 18's lumen 110 can be a first lumen of the delivery assistance tube and the delivery assistance tube can comprise a second lumen 118 that is surrounded by the first lumen and extends from the delivery assistance tube's proximal end 94a to the delivery assistance tube's distal end 94b such that the delivery assistance tube's distal end includes an opening 122; because the second lumen extends to the delivery assistance tube's proximal end, any gas that may enter the second lumen through the opening in the tube's distal end cannot enter covered stent 14's lumen 34 when pressure is reduced at the delivery assistance tube's proximal end. In such embodiments, second lumen 118 can be configured to receive, for example, a guide wire such that delivery assistance tube 18 can be advanced through a patient's vasculature along a guide wire extending through the second lumen.

Delivery assistance tube 18's lumen 110 can be sized such that enough gas can be readily evacuated therefrom with, for example, a syringe to create a pressure differential that can counteract the radially-outward urging force of covered stent 14's frame 26. For example, an internal transverse dimension 114 (e.g., diameter) of lumen 110 can be less than or equal to any one of, or between any two of, 0.18, 0.17, 0.16, 0.15, 0.14, 0.13, 0.12, 0.11, 0.10, 0.09, or 0.08 cm (e.g., less than or equal to 0.14 cm) and the lumen can have a volume that is less than or equal to any one of, or between any two of, 2, 1.9, 1.8, 1.7, 1.6, 1.5, 1.4, 1.3, 1.2, 1.1, or 1.0 cubic centimeters (cm³) (e.g., less than or equal to 1.5 cm³). Moreover, a collective area of openings 102—which can affect the magnitude of the radially-inward force resulting from the pressure differential—can be sufficient to allow the pressure reduction to counteract the radially-outward urging force of covered stent 14's frame 26. For example, a collective area of openings 102 can be greater than or equal to any one of, or between any two of, 20%, 30%, 40%, 50%, 60%, 70%, or 80% (e.g., at least 40%) of a surface area of an inner surface of covered stent 14's gas-impermeable membrane 30 when the covered stent is in the compressed state.

Delivery assistance tube 18 can have a length 124 that allows it to be advanced through a patient's vasculature to a desired location while its distal portion 98 is disposed in covered stent 14's lumen 34 and while maintaining access to the delivery assistance tube's proximal end 94a. Such access to delivery assistance tube 18's proximal end 94a can allow a user to control the pressure at the proximal end and thus within covered stent 14's lumen 34 that, via the delivery assistance tube's openings 102 and lumen 110, is in fluid communication with the delivery assistance tube's proximal end. For example, to allow advancement from an insertion point at the groin to the ICA or MCA, delivery assistance tube 18's length 124, measured between proximal and distal ends 94a and 94b of the delivery assistance tube, can be greater than or equal to any one of, or between any two of, 90, 100, 110, 120, 130, 140, 150, or 160 cm (e.g., at least 120 cm).

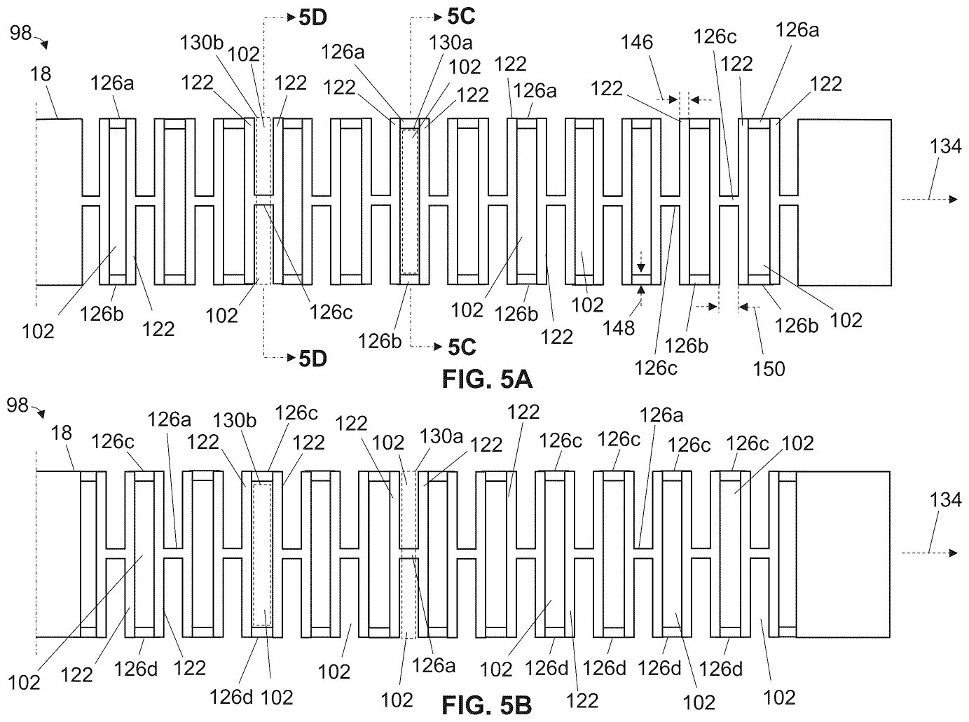

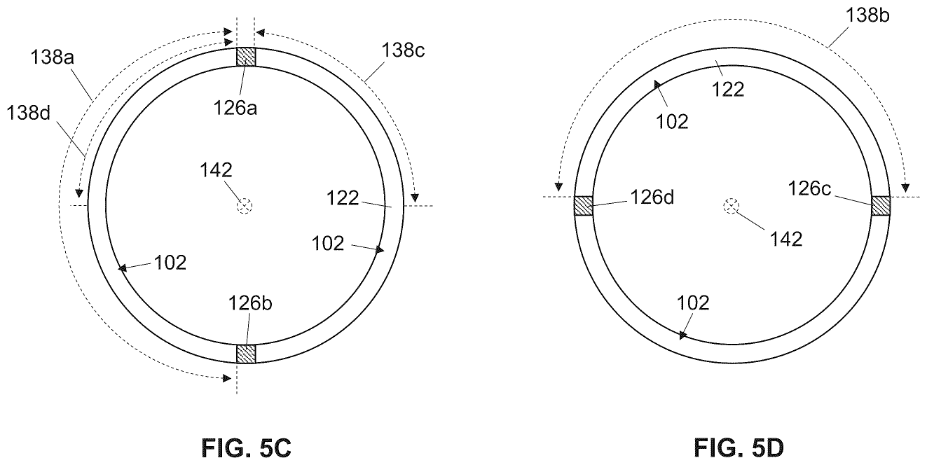

Delivery assistance tube 18 is preferably relatively flexible such that it can be readily advanced through a patient's vasculature. For example, delivery assistance tube 18 can comprise an elastic material such as nitinol that promotes its flexibility. Referring additionally to FIGS. 5A-5D, delivery assistance tube 18's distal portion 98 can have an opening-defining geometry that, in addition to allowing a pressure reduction at the delivery assistance tube's proximal end 94a to draw covered stent 14 radially inward when the delivery assistance tube's distal portion is disposed in the covered stent's lumen 34, can further promote the flexibility of the distal portion and accordingly facilitate the delivery assistance tube's advancement through the tortuous vasculature of a patient. As shown, delivery assistance tube 18's openings 102 can comprise a plurality of slits, which can each extend in a circumferential direction that is substantially perpendicular to a longitudinal axis 142 that extends between proximal and distal ends 94a and 94b of the delivery assistance tube. To define slits 102, the outer wall of delivery assistance tube 18 can include, in distal portion 98, a plurality of circumferential segments 122 that each extend in the circumferential direction and a plurality of axial segments 126a-126d that each extend between two of the circumferential segments in an axial direction 134 that is substantially parallel to longitudinal axis 142, where each of a plurality (up to and including approximately all) of the slits can be circumscribed by two of the axial segments and two of the circumferential segments. To promote flexibility, a width 146 of each of circumferential segments 142 (e.g., measured along axial direction 134) and a width 148 of each of axial segments 126a-126d (e.g., measured along a radial direction) can each be relatively small, such as less than or equal to any one of, or between any two of, 0.10, 0.09, 0.08, 0.07, 0.06, 0.05, 0.04, 0.03, or 0.02 cm (e.g., less than or equal to 0.05 cm). Each of slits 102 can likewise be relatively narrow to promote a balance between available slit area for exerting a radially-inward force on covered stent 14 and the strength of distal portion 98; for example, each slit can have a width 150 (e.g., measured along axial direction 134) that is less than or equal to any one of, or between any two of, 0.10, 0.09, 0.08, 0.07, 0.06, 0.05, 0.04, 0.03, or 0.02 cm, which can optionally be approximately the same as width 146 of each of circumferential segments 142 and/or as width 148 of each of axial segments 126a-126d.

To promote multi-directional flexibility, there can be multiple subsets of axial segments 126a-126d having different circumferential positions. For example, as shown, axial segments 126a-126d can include a plurality of first axial segments 126a, a plurality of second axial segments 126b, a plurality of third axial segments 126c, and a plurality of fourth axial segments 126d that are orthogonally-spaced, e.g., where, taken about longitudinal axis 142, an angular separation 138a between each of the first axial segments and each of the second axial segments is approximately 180° (FIG. 5C), an angular separation 138b between each of the third axial segments and each of the fourth axial segments is approximately 180° (FIG. 5D), and an angular separation 138c between each of the first axial segments and each of the third axial segments and an angular separation 138d between each of the first axial segments and each of the fourth axial segments are each approximately 90° (FIG. 5C). Taken in a radial direction that is perpendicular to longitudinal axis 142, each of first axial segments 126a can be disposed across from a respective one of second axial segments 126b and each of third axial segments 126c can be disposed across from a respective one of fourth axial segments 126d such that there are a plurality of pairs of first and second axial segments and a plurality of pairs of third and fourth axial segments, where along axial direction 134 there is an alternating sequence of pairs of first and second axial segments and pairs of third and fourth axial segments. As a result, slits 102 can include a plurality of first pairs 130a of slits and a plurality of second pairs 130b of slits; FIG. 5C is a sectional view taken about a line extending through one of the first pairs of slits and FIG. 5D is a sectional view taken about a line extending through one of the second pairs of slits. As shown, for each of first pairs 130a of slits 102, two of circumferential segments 122, one of first axial segments 126a, and one of second axial segments 126b can circumscribe each of the slits of the first pair, and for each of second pairs 130b of slits, two of the circumferential segments, one third axial segment 126c, and one of fourth axial segments 126d can circumscribe each of the slits of the second pair. With the alternating pairs of first and second axials segments 126a and 126b and pairs of third and fourth axial segments 126c and 126d, taken in axial direction 134, each of first pairs 130a of slits 102 can be adjacent to at least one of second pairs 130b of slits. This geometry can promote distal portion 98's flexibility in multiple directions that are perpendicular to axial direction 134 such that it can be readily advanced through a patient's vasculature.

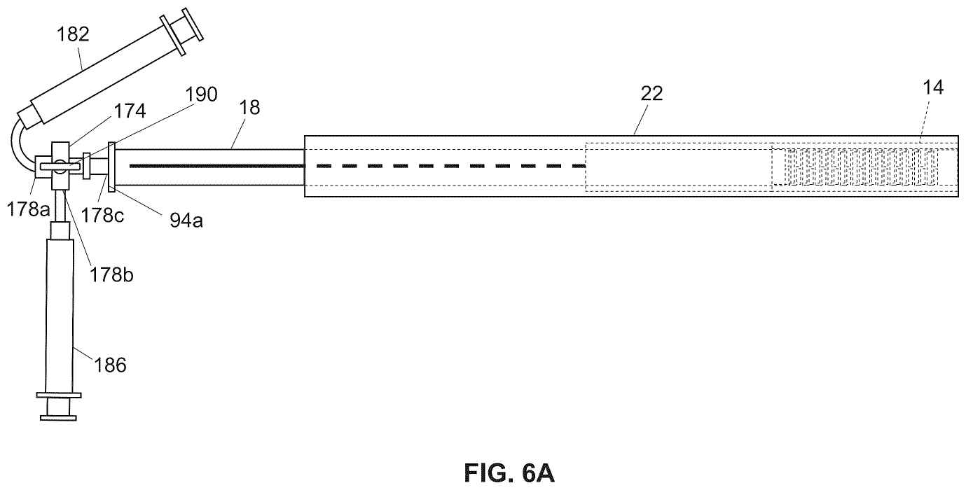

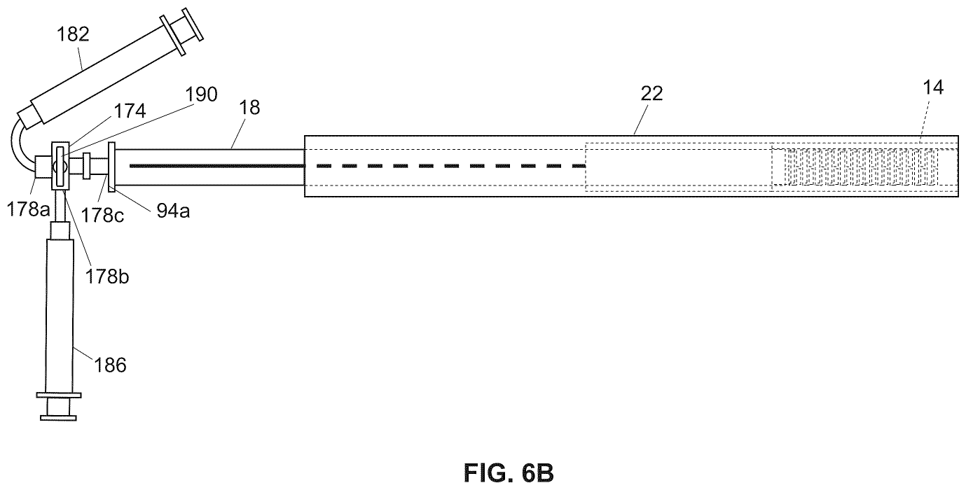

Delivery assistance tube 18 can be configured to be coupled to a negative pressure source such that the negative pressure source can reduce pressure at the delivery assistance tube's proximal end 94a and accordingly cause the gas evacuation through the delivery assistance tube's lumen 110 that yields a pressure differential to urge covered stent 14 radially inward. Referring to FIGS. 6A and 6B, a negative pressure source 182 can be coupled to delivery assistance tube 18's proximal end 94a via a valve 174. Valve 174 can have first, second, and third ports 178a-178c and can be changeable between first and second states by, for example, turning a handle 190 of the valve, where in the first state third port 178c can be in fluid communication with first port 178a but not with second port 178b (FIG. 6A) and in the second state the third port can be in fluid communication with the second port but not with the first port (FIG. 6B). Third port 178c of valve 174 can be coupled to delivery assistance tube 18's proximal end 94a and negative pressure source 182 can be coupled to first port 178a of the valve such that the negative pressure source is in fluid communication with the delivery assistance tube's lumen 110 when the valve is in the first state, allowing the negative pressure source to reduce pressure at the delivery assistance tube's proximal end and thus evacuate gas through the delivery assistance tube's lumen to reduce pressure in the delivery assistance tube's distal portion 98 and in covered stent 14's lumen 34 when the distal portion is disposed in the covered stent's lumen. As shown, negative pressure source 182 can comprise a syringe configured to withdraw sufficient gas from lumen 110 to yield the pressure reduction needed to counteract the radially-outward urging of covered stent 14's frame 26; for example, a capacity of the syringe can be greater than or equal to any one of, or between any two of, 20, 21, 22, 23, 24, 25, 26, 27, 28, 29, 30, 31, or 32 cm³ (e.g., at least 25 cm³). In other embodiments, negative pressure source 182 can comprise a pump, such as an electromechanical pump. When negative pressure source 182 is in fluid communication with delivery assistance tube 18's lumen 110 and the delivery assistance tube's distal portion 98 is disposed in lumen 34 of covered stent 14 (e.g., while the covered stent is in the compressed state), the negative pressure source can be configured to reduce pressure within the covered stent's lumen by greater than or equal to any one of, or between any two of, 70, 75, 80, 85, 90, 95, 100, or 105 kPa (e.g., at least 90 kPa).

Delivery assistance tube 18's proximal end 94a can also be coupled to a positive pressure source 186 like a syringe or pump, such as through valve 174 whose second port 178b can be coupled to the positive pressure source such that the positive pressure source is in fluid communication with the delivery assistance tube's lumen 110 when the valve is in the second state. Positive pressure source 186 can be configured to increase pressure in delivery assistance tube's lumen 110 and thus in lumen 34 of covered stent 14—such as by introducing liquid therein—after the pressure reduction such that the covered stent, through at least the radially-outward urging from its frame 26, can expand from the compressed state to the expanded state for deployment. While as shown two separate pressure sources—negative pressure source 182 and positive pressure source 186—can both be coupled to delivery assistance tube 18's proximal end 94a, in other embodiments the negative pressure source can also serve as a positive pressure source (e.g., can be configured to both decrease and increase pressure at the delivery assistance tube's proximal end).

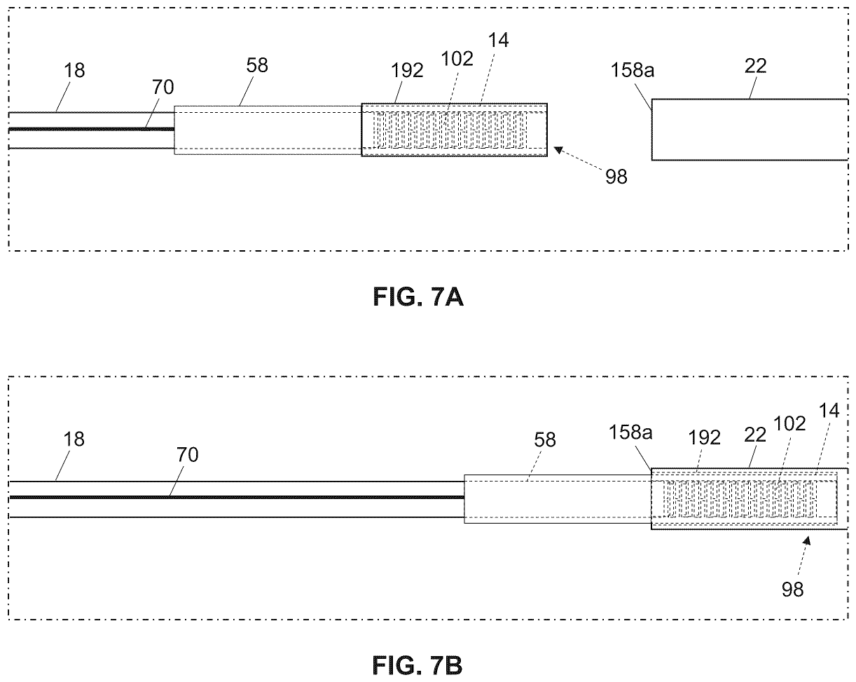

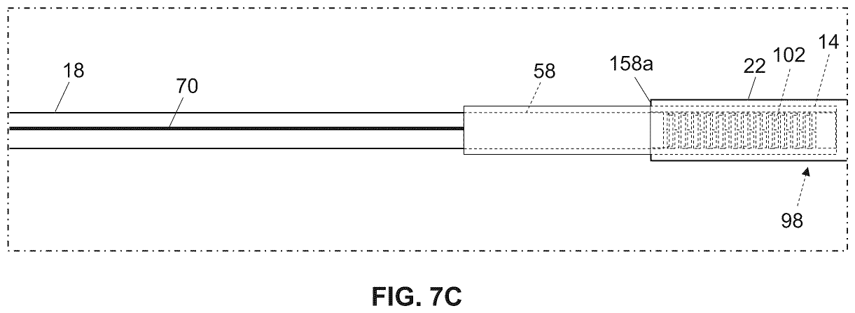

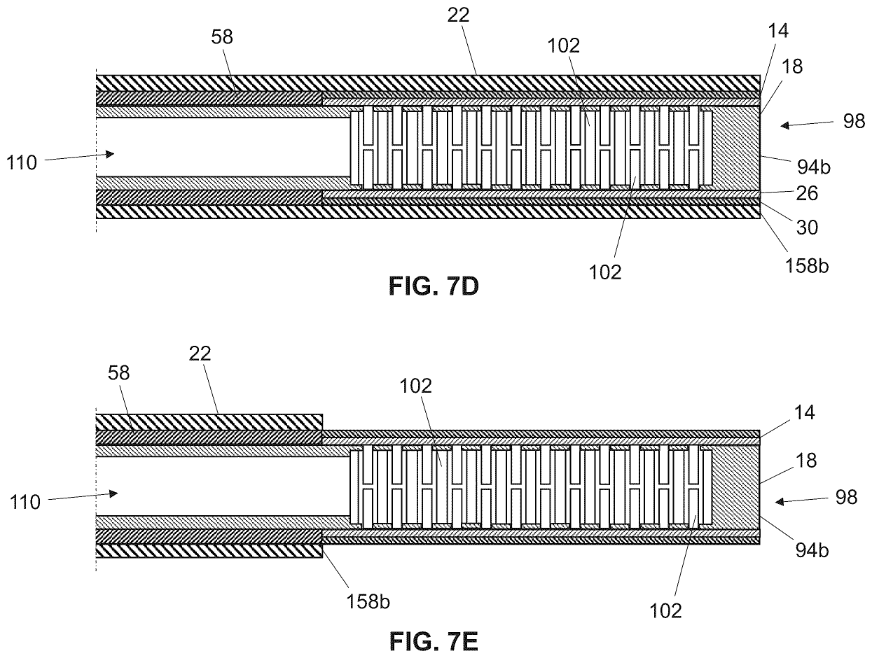

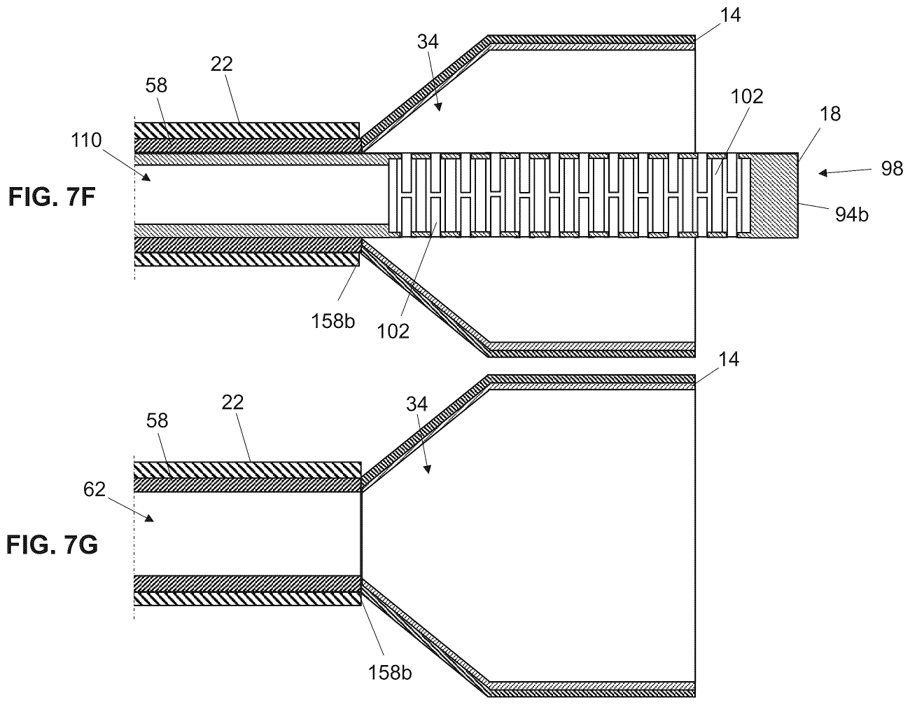

Referring to FIGS. 7A-7G, shown is a procedure by which covered stent 14 can be advanced through first guide tube 22 in the compressed state and deployed therefrom to expand to the expanded state when using delivery assistance tube 18. Before reducing pressure at proximal end 94a of delivery assistance tube 18 and before introducing covered stent 14 into first guide tube 22's lumen 154, the covered stent can be disposed in an introducer tube 192 such as a peel-away sheath or dilator and the delivery assistance tube's distal portion 98 can be disposed in the covered stent's lumen 34 (FIG. 7A). In this configuration, introducer tube 192 can help maintain covered stent 14 in the compressed state such that its gas-impermeable membrane 30 can engage with delivery assistance tube 18's distal portion 98 and create a seal with openings 102. Pressure can then be reduced in distal portion 98 of delivery assistance tube 18 to create a pressure differential that urges covered stent 14 radially inward and counteracts the radially-outward urging force generated by the covered stent's frame 26, e.g., by reducing pressure at the delivery assistance tube's proximal end 94a using negative pressure source 182 that can be coupled to the delivery assistance tube's proximal end via, for example, valve 174 as described above. After pressure is reduced, introducer tube 192 can be inserted into first guide tube 22's lumen 154 while covered stent 14 is disposed in the introducer tube and delivery assistance tube 18's distal portion 98 is disposed the covered stent's lumen 34 (FIG. 7B), and the introducer tube can thereafter be removed from the first guide tube's lumen, leaving the covered stent and at least the distal portion of the delivery assistance tube disposed in the first guide tube's lumen (FIG. 7C).

With covered stent 14 introduced into first guide tube 22's lumen 154 and urged radially inward around distal portion 98 of delivery assistance tube 18, the covered stent and delivery assistance tube can be advanced through the first guide tube's lumen toward the covered stent's distal end 158b (FIG. 7D). To deploy covered stent 14, the covered stent can be positioned relative to first guide tube 22 such that at least a portion of the covered stent is disposed distally of distal end 158b of the first guide tube (FIG. 7E), such as by retracting the first guide tube and/or advancing the covered stent. When covered stent 14 is so-positioned, pressure within lumen 34 of the covered stent can be increased so that there is no longer a pressure differential urging the covered stent radially inward around distal portion 98 of delivery assistance tube 18, and the covered stent's frame 26 can accordingly urge the covered stent radially outward to the expanded state (FIG. 7F). Delivery assistance tube 18 can thereafter be removed from covered stent 14's lumen 34 and first guide tube 22's lumen 154 (FIG. 7G) such that there is a pathway for a thrombus to be ingested through the covered stent and removed proximally toward the first guide tube's proximal end 158a.

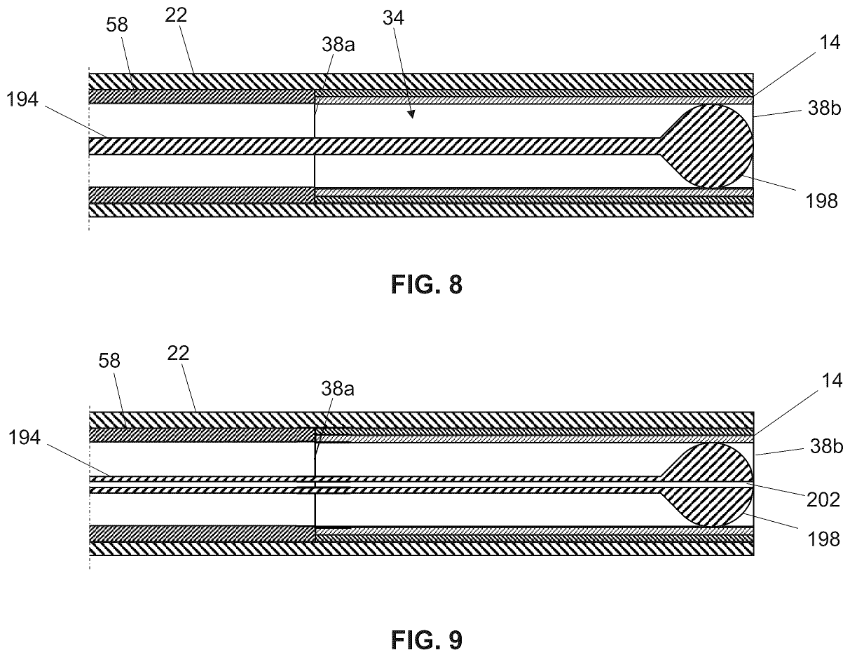

Referring to FIGS. 8 and 9, in some embodiments pressure can be reduced within lumen 34 of covered stent 14 to urge the covered stent radially inward toward the compressed state using a mechanism other than delivery assistance tube 18. For example, the embodiments shown in FIGS. 8 and 9 can each comprise a pusher 194 that is configured to be received in lumen 34 of covered stent 14 and a compliant bead 198 that is coupled to the pusher and configured to sealingly engage a distal portion of an inner surface of the covered stent when the covered stent is in the compressed state such that gas is not permitted to flow from a distal end 98b of the covered stent to a portion of the covered stent's lumen that is proximal of the compliant bead. With such a seal created by compliant bead 198, reducing pressure at a location that is proximal of the compliant bead can cause gas to evacuate from covered stent 14's lumen 34 to reduce the pressure therein and accordingly yield a pressure differential that urges the covered stent radially inward. To illustrate, with aspiration tube 58 coupled to proximal end 38a of covered stent 14, pressure can be reduced at the aspiration tube's proximal end 66a to evacuate gas from and accordingly reduce pressure in the covered stent's lumen 34 (e.g., using a negative pressure source like a syringe or pump coupled to the aspiration tube's proximal end, optionally with the same valve-based arrangement with which delivery assistance tube 18's proximal end 94a can be coupled to a negative pressure source (and optionally a positive pressure source) via a valve). Lumen 62 of aspiration tube 58 can have any suitable volume to readily allow enough gas evacuation to yield a pressure reduction that can urge covered stent 14 radially inward, such as a volume that is less than or equal to any one of, or between any two of, 2, 1.9, 1.8, 1.7, 1.6, 1.5, 1.4, 1.3, 1.2, 1.1, or 1.0 cm³ (e.g., less than or equal to 1.5 cm³). Pusher 194 can allow compliant bead 198 to be inserted into and withdrawn from covered stent 14's lumen 34, and can comprise, for example, a guide wire or rod (FIG. 8) or a microcatheter where a lumen 202 extends from a proximal end of the microcatheter through bead 198 such that a guide wire can pass through the microcatheter and bead, allowing advancement over a guide wire in a patient's vasculature (FIG. 9). Pusher 194 can be more flexible than delivery assistance tube 18 and thus may better facilitate the system's advancement through a patient's vasculature.

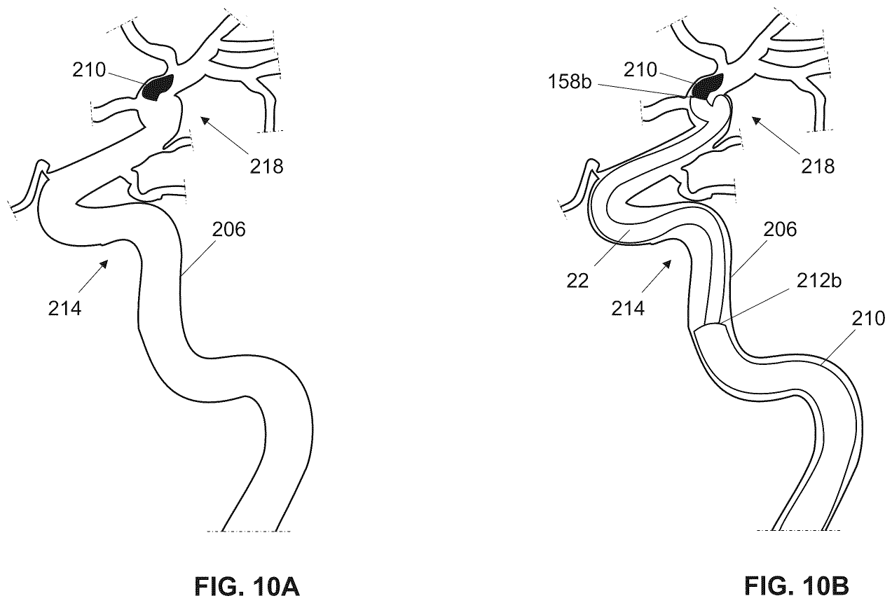

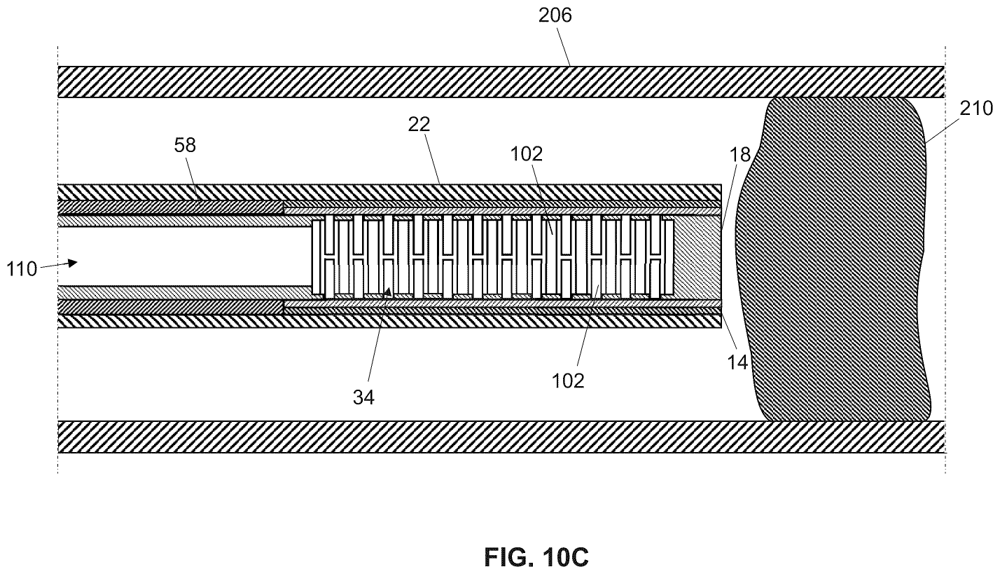

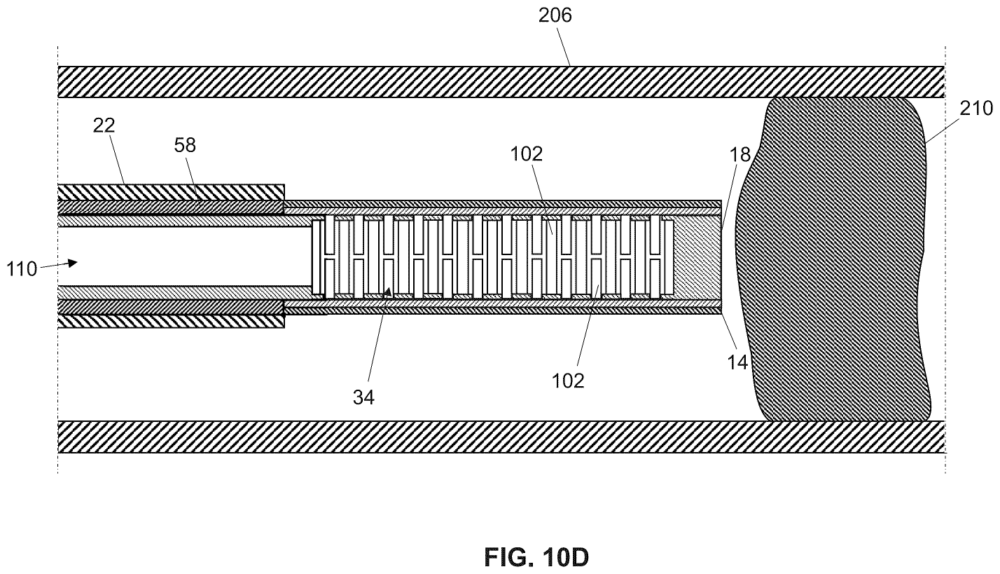

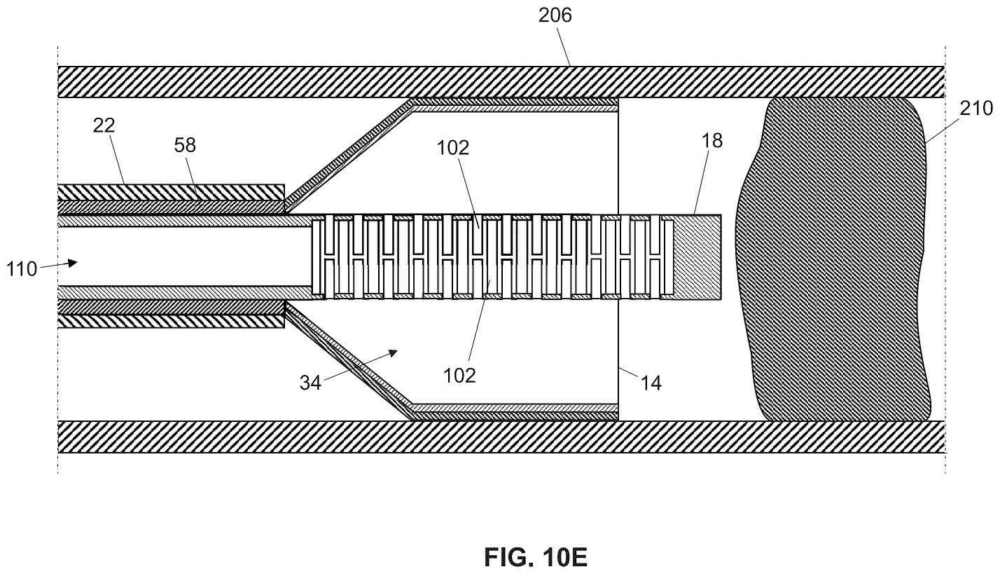

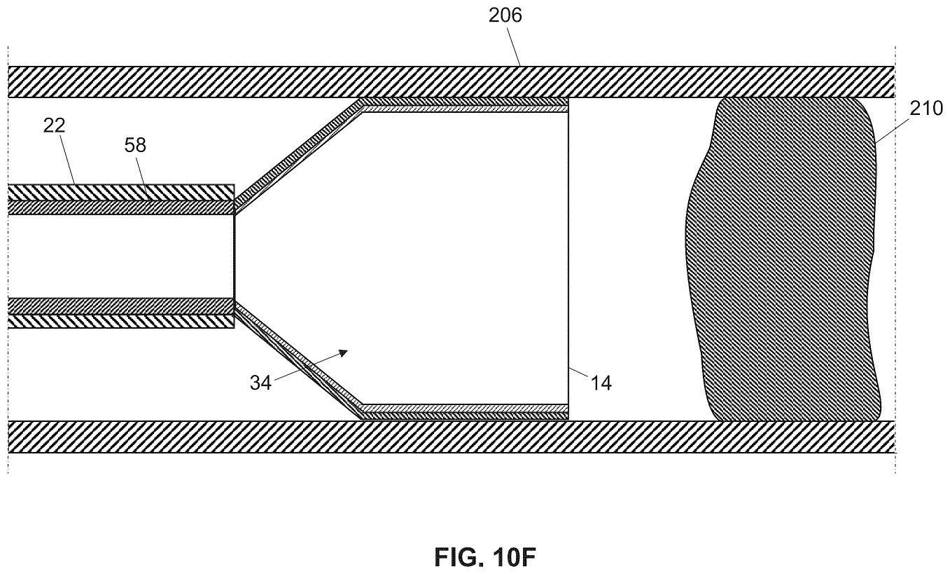

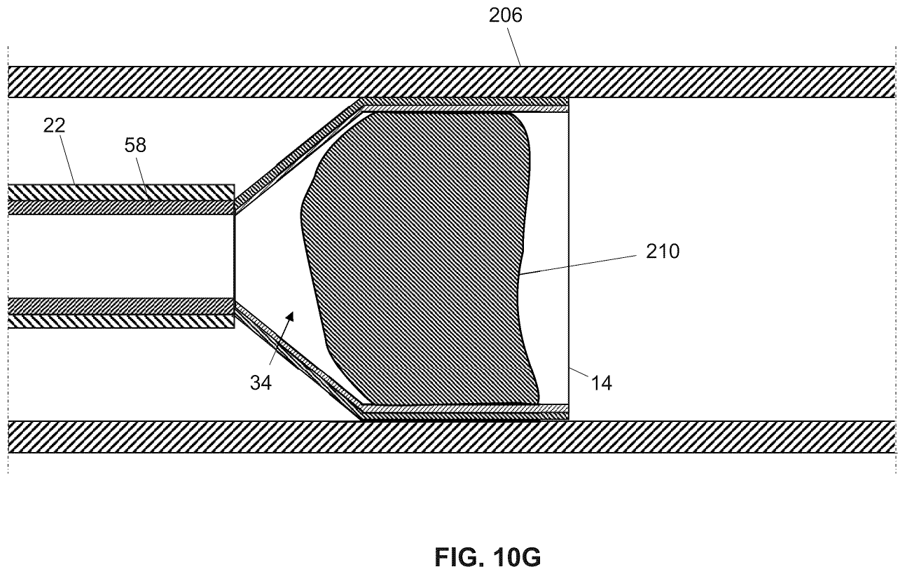

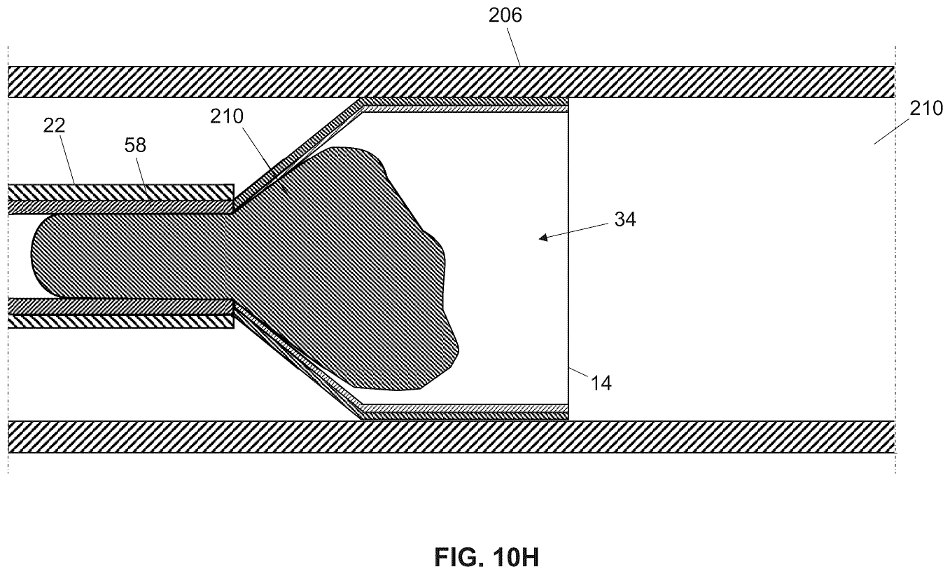

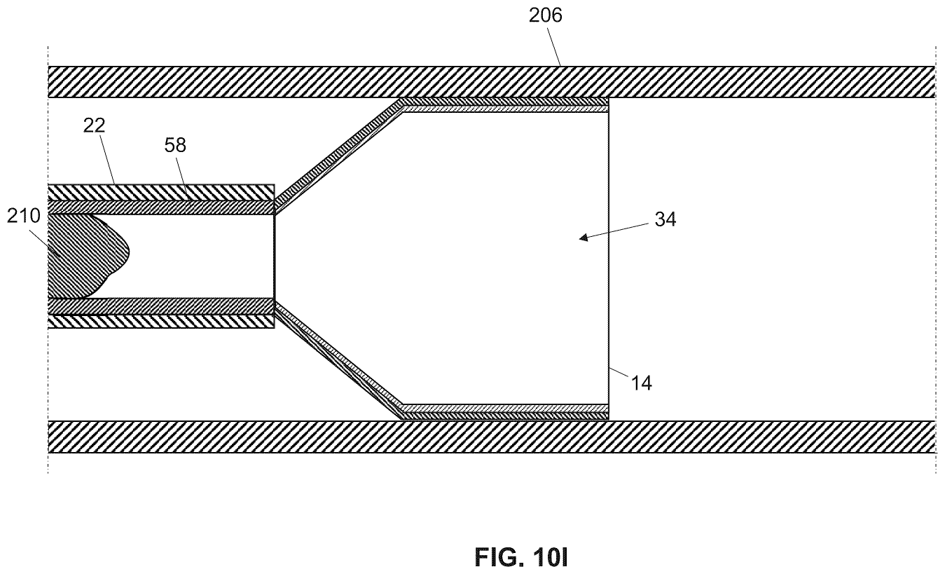

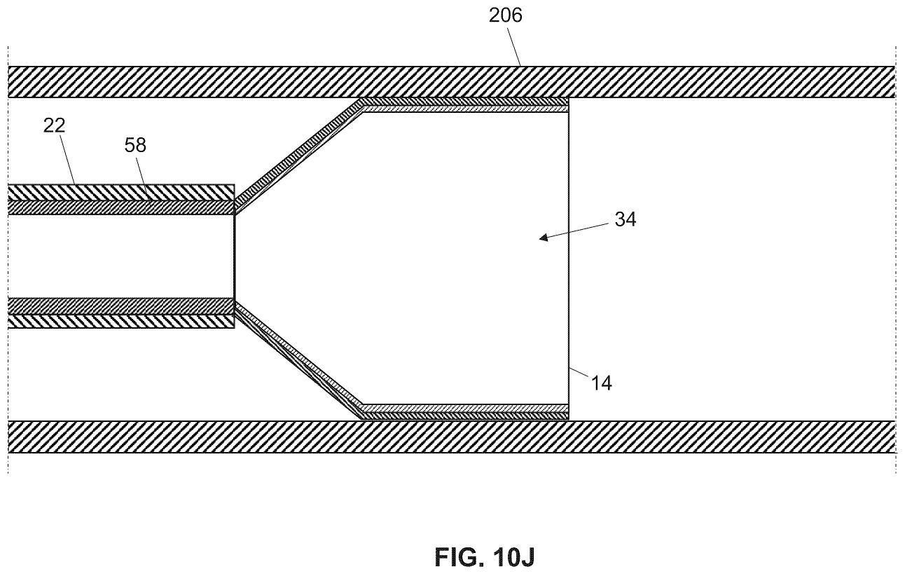

Referring to FIGS. 10A-10J, illustrated are some of the present methods for thrombus removal. As shown, a thrombus (e.g., 210) can be disposed in the vasculature (e.g., 206) of a patient, such as in the patient's ICA (e.g., 214) or MCA (e.g., 218) (e.g., the M1 segment thereof) (FIG. 10A). Some methods comprise advancing a first guide tube (e.g., 22) (e.g., any of those described above) through the patient's vasculature, optionally such that the distal end (e.g., 158b) of the first guide tube is disposed in the ICA or the MCA (e.g., when that is where the thrombus is located) (FIG. 10B). Some of the present systems can comprise a second guide tube (e.g., 210) that is substantially the same as the first guide tube, the primary exception being that the second guide tube's lumen is larger than the first guide tube's lumen such that the second guide tube's lumen is configured to receive the first guide tube. For example, an internal transverse dimension (e.g., diameter) of the second guide tube's lumen can be greater than or equal to any one of, or between any two of, 0.18, 0.19, 0.20, 0.21, 0.22, 0.23, 0.24, 0.25, or 0.26 cm (e.g., at least 0.22 cm) (e.g., the second guide tube can be an 8 F catheter or a 6 F sheath). As shown in FIG. 10B, for some methods using one of such systems, the second guide tube can be advanced through the vasculature of the patient and advancing the first guide tube through the vasculature of the patient can comprise advancing the first guide tube through the second guide tube and beyond a distal end (e.g., 212b) of the second guide tube. The second guide tube can accordingly facilitate advancement of the first guide tube through a portion of the vasculature until a point past which it can be difficult to advance the larger and thus less-flexible second guide tube. For example, when the thrombus is in the ICA or the MCA, the distal end of the larger second guide tube can be positioned proximally of the ICA and/or MCA, with the smaller first guide tube being advanced past the distal end of the second guide tube to the ICA and/or MCA to reach the thrombus.

Some methods comprise reducing pressure within a lumen (e.g., 34) of a covered stent (e.g., 14) (e.g., any of those described above) such that a pressure differential urges the covered stent radially inward in the compressed state and, while pressure within the lumen of the covered stent is reduced and the covered stent is in the compressed state, advancing the covered stent through the first guide tube (FIG. 10C). Reducing pressure within the lumen of the covered stent can be performed such that the pressure within the lumen decreases by greater than or equal to any one of, or between any two of, 70, 75, 80, 85, 90, 95, 100, or 105 kilopascals (kPa) (e.g., at least 90 kPa). The pressure reduction and advancement can be achieved in any of the manners described above. For example, when using a delivery assistance tube (e.g., 18) (e.g., any of those described above), pressure can be reduced at least by positioning a distal portion (e.g., 98) of the delivery assistance tube within the covered stent's lumen—where the distal portion comprises the delivery assistance tube's distal end (e.g., 94b) and a plurality of openings (e.g., 102) that extend through an outer wall of the delivery assistance tube—and reducing pressure at a proximal end (e.g., 94a) of the delivery assistance tube while the covered stent is in the compressed state such that gas flows from the distal portion of the delivery assistance tube to the proximal end of the delivery assistance tube through a lumen (e.g., 110) of the delivery assistance tube (e.g., using a negative pressure source (e.g., 182) like a syringe or pump). Or, when using a pusher (e.g., 194) with a compliant bead (e.g., 198) coupled thereto, the compliant bead can be positioned in the lumen of the covered stent such that the compliant bead forms a seal with the covered stent's inner surface in a distal portion thereof and pressure can be reduced at a proximal end (e.g., 66a) of an aspiration tube (e.g., 58) coupled to a proximal end (e.g., 38a) of the covered stent such that gas flows from the covered stent's lumen toward the aspiration tube's proximal end. As explained above, the pressure can be reduced before the covered stent is disposed in the lumen of the first guide tube, which can optionally receive the covered stent while the covered stent is disposed in an introducer tube (e.g., 192) that can be removed from the first guide tube after the covered stent is received in the first guide tube's lumen. The covered stent can thereafter be advanced through the first guide tube (e.g., while the delivery assistance tube's distal portion or the compliant bead is disposed in the covered stent's lumen).

Some methods comprise, after advancing the covered stent through the first guide tube, expanding the covered stent from the compressed state to the expanded state in the vasculature of the patient (FIGS. 10D and 10E). As explained above, this can comprise positioning the covered stent relative to the first guide tube such that at least a portion of the covered stent is disposed distally of a distal end of the first guide tube (FIG. 10D) (e.g., by retracting the first guide tube by pulling it proximally and/or advancing the covered stent by pushing one or more of the extension component(s) (e.g., 54) of the covered stent's stent assembly) and increasing pressure within the lumen of the covered stent such that the covered stent's frame (e.g., 26) can urge the covered stent radially outward to the expanded state (FIG. 10E). Increasing pressure in the covered stent's lumen can comprise introducing a liquid such as a saline solution into the lumen of the covered stent using, for example, a positive pressure source (e.g., 186) like a syringe or pump. To illustrate, when the delivery assistance tube is used, liquid can be introduced into the delivery assistance tube's lumen through its proximal end and the liquid can flow to the delivery assistance tube's distal portion and through its openings to enter the covered stent's lumen. When the pusher with the compliant bead is used, the liquid can be introduced into the aspiration tube's lumen through its proximal end and thereafter flow to the covered stent's lumen. In the expanded state, the covered stent can engage the patient's vasculature and occlude blood flow proximally and distally of the covered stent.

Some methods comprise withdrawing one or more components used to reduce pressure within the lumen of the covered stent (FIG. 10F)—such as the delivery assistance tube or the pusher having a compliant bead coupled thereto—and aspirating the thrombus into the distal end of the covered stent and through the lumen of the covered stent while the covered stent is in the expanded state (FIGS. 10G-10J). Aspirating the thrombus can include reducing pressure at the proximal end of the first guide tube and/or the optional aspiration tube (e.g., using a syringe or pump), yielding a negative pressure differential between the covered stent's distal end and the first guide tube's proximal end and/or aspiration tube's proximal end that can cause the thrombus to aspirate into the covered stent. Because the covered stent is in the expanded state, such ingestion can readily occur. The thrombus, after passing through the covered stent's lumen, can continue proximally through one or more tubes of the system—such as the optional aspiration tube, the first guide tube, and/or the second guide tube—for removal from the vasculature.

Some of the present stent delivery systems comprise a covered stent that comprises a lumen, and some of the present methods for use in thrombus removal comprise reducing pressure within a lumen of a covered stent. In some embodiments, the covered stent is radially expandable from a compressed state to an expanded state, wherein a maximum transverse dimension of the lumen of the covered stent, measured perpendicularly to a longitudinal axis extending between proximal and distal ends of the covered stent, is larger when the covered stent is in the expanded state than when the covered stent is in the compressed state.

The covered stent, in some embodiments, comprises a frame configured to urge the covered stent toward the expanded state when the stent is in the compressed state. In some embodiments, the frame is configured to urge the covered stent toward the expanded state with a force at which, when the covered stent is in the compressed state and a surface surrounds the covered stent, a pressure that the covered stent exerts on the surface is at least 85 kilopascals (kPa). The frame, in some embodiments, comprises nitinol.

In some embodiments, the covered stent comprises a gas-impermeable membrane coupled to the frame. The lumen of the covered stent, in some embodiments, is surrounded by the gas-impermeable membrane.

Some stent delivery systems comprise a delivery assistance tube that comprises a distal portion configured to be positioned within the lumen of the covered stent, and in some methods reducing pressure within the lumen of the covered stent comprises positioning a distal portion of a delivery assistance tube within the lumen of the covered stent. In some embodiments, the distal portion of the delivery assistance tube comprises a distal end of the delivery assistance tube and a plurality of openings that extend through an outer wall of the delivery assistance tube. A collective area of the openings, in some embodiments, is at least 40% of a surface area of an inner surface of the gas-impermeable membrane. The delivery assistance tube, in some embodiments, comprises a lumen that extends from a proximal end of the delivery assistance tube to the distal portion of the delivery assistance tube and is in fluid communication with the openings. In some systems, the distal portion of the delivery assistance tube is positioned in the lumen of the covered stent and the proximal end of the delivery assistance tube is coupled to a negative pressure source such that the negative pressure source is in fluid communication with the lumen of the delivery assistance tube. In some methods, reducing pressure within the lumen of the covered stent comprises reducing pressure at a proximal end of the delivery assistance tube such that gas flows from the distal portion of the delivery assistance tube to the proximal end of the delivery assistance tube through a lumen of the delivery assistance tube. Reducing pressure within the lumen of the covered stent, in some methods, is performed such that the pressure within the lumen decreases by at least 90 kilopascals (kPa).

In some embodiments, the openings of the distal portion of the delivery assistance tube comprise a plurality of slits that each extend in a circumferential direction that is substantially perpendicular to a longitudinal axis that extends between the proximal and distal ends of the delivery assistance tube. In the distal portion of the delivery assistance tube, in some embodiments, the outer wall of the delivery assistance tube includes a plurality of circumferential segments that each extend in the circumferential direction and a plurality of axial segments that each extend between two of the circumferential segments in an axial direction that is substantially parallel to the longitudinal axis that extends between the proximal and distal ends of the delivery assistance tube. In some embodiments, the axial segments include a plurality of first axial segments, a plurality of second axial segments, a plurality of third axial segments, and a plurality of fourth axial segments. In some embodiments, an angular separation, taken about the longitudinal axis that extends between the proximal and distal ends of the delivery assistance tube, between each of the first axial segments and each of the second axial segments is approximately 180°, between each of the third axial segments and each of the fourth axial segments is approximately 180°, and/or between each of the first axial segments and each of the third and fourth axial segments is approximately 90°. The slits, in some embodiments, include a plurality of first pairs of slits and a plurality of second pairs of slits. In some embodiments, for each of the first pairs of slits, two of the circumferential segments, one of the first axial segments, and one of the second axial segments circumscribe each of the slits of the first pair. For each of the second pairs of slits, in some embodiments, two of circumferential segments, one of the third axial segments, and one of the fourth axial segments circumscribe each of the slits of the second pair. In some embodiments, taken in the axial direction, each of the first pairs of slits is adjacent to at least one of the second pairs of slits.

In some embodiments, the distal end of the delivery assistance tube is closed. In other embodiments, the lumen of the delivery assistance tube is a first lumen of the delivery assistance tube and the delivery assistance tube comprises a second lumen that extends from the proximal end of the delivery assistance tube to the distal end of the delivery assistance tube such that the distal end of the delivery assistance tube includes an opening. The first lumen, in some embodiments, surrounds the second lumen.

The delivery assistance tube, in some embodiments, comprises nitinol.

Some systems comprise a pusher configured to be received in the lumen of the covered stent and a compliant bead that is coupled to the pusher. The compliant bead, in some systems, is configured to sealingly engage an inner surface of a distal portion of the covered stent when the covered stent is in the compressed state such that gas is not permitted to flow from the distal end of the covered stent to a portion of the lumen of the covered stent that is proximal of the compliant bead.

Some systems comprise an aspiration tube coupled to the proximal end of the covered stent. In some systems that comprise the delivery assistance tube, the delivery assistance tube is configured to move axially within a lumen of the aspiration tube. In some systems comprising the pusher, the pusher is configured to move axially within a lumen of the aspiration tube. In some of such embodiments, the covered stent is in the compressed state, the pusher is disposed in the lumen of the aspiration tube and the lumen of the covered stent, the compliant bead is sealingly engaged with the inner surface of the distal portion of the covered stent, and a proximal end of the aspiration tube is coupled to a negative pressure source such that the negative pressure source is in fluid communication with the lumen of the aspiration tube. In some systems, a volume of the lumen of the aspiration tube is less than or equal to 2 cubic centimeters (cm³) and/or a maximum external transverse dimension of the aspiration tube, measured perpendicularly to a longitudinal axis extending between proximal and distal ends of the tube, is less than or equal to 1.6 millimeters (mm).

Some systems comprise a guide tube. The covered stent, in some systems, is configured to move axially within a lumen of the guide tube. In some systems, a maximum transverse dimension of the guide tube, measured perpendicularly to a longitudinal axis that extends between proximal and distal ends of the guide tube, is less than or equal to 2.2 millimeters (mm).

Some methods comprise advancing a first guide tube through vasculature of a patient and while pressure within the lumen of the covered stent is reduced and while the covered stent is in the compressed state, advancing the covered stent through the first guide tube. Some methods comprise advancing a second guide tube through the vasculature of the patient such that a distal end of the second guide tube is positioned proximally of an internal carotid artery and/or a middle cerebral artery of the patient. In some of such methods, advancing the first guide tube through the vasculature of the patient comprises advancing the first guide tube through the second guide tube and beyond the distal end of the second guide tube such that the distal end of the first guide tube is positioned in the internal carotid artery or the middle cerebral artery.

Some methods comprise, after advancing the covered stent through the first guide tube, expanding the covered stent from the compressed state to the expanded state in the vasculature of the patient. In some methods, expanding the covered stent from the compressed state to the expanded state includes positioning the covered stent relative to the first guide tube such that at least a portion of the covered stent is disposed distally of a distal end of the first guide tube and increasing pressure within the lumen of the covered stent. In some methods, increasing pressure within the lumen of the covered stent comprises introducing a liquid into the lumen of the covered stent.

Some methods comprise aspirating a thrombus into the distal end of the covered stent and through the lumen of the covered stent while the covered stent is in the expanded state.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The terms “substantially,” “about,” and “approximately” are each defined as largely but not necessarily wholly what is specified—and include what is specified, e.g., substantially 90 degrees includes 90 degrees and substantially parallel includes parallel—as understood by a person of ordinary skill in the art. As used herein, “substantially parallel” means within 10 degrees of parallel to, and “substantially perpendicular” means within 10 degrees of perpendicular to. In any disclosed embodiment, the terms “approximately” and “about” may each be substituted with “within [a percentage] of” what is specified, where the percentage includes 0.1, 1, 5, and 10 percent.

The terms “comprise” and any form thereof such as “comprises” and “comprising,” “have” and any form thereof such as “has” and “having,” and “include” and any form thereof such as “includes” and “including” are open-ended linking verbs. As a result, an apparatus or system that “comprises,” “has,” or “includes” one or more elements possesses those one or more elements but is not limited to possessing only those elements. Likewise, a method that “comprises,” “has,” or “includes” one or more steps possesses those one or more steps but is not limited to possessing only those one or more steps.

Any embodiment of any of the apparatuses, systems, and methods can consist of or consist essentially of—rather than comprise/have/include—any of the described steps, elements, and/or features. Thus, in any of the claims, the term “consisting of” or “consisting essentially of” can be substituted for any of the open-ended linking verbs recited above in order to change the scope of a given claim from what it would otherwise be using the open-ended linking verb.

Further, an apparatus or system that is configured in a certain way is configured in at least that way, but it can also be configured in other ways than those specifically described.

The feature or features of one embodiment may be applied to other embodiments, even though not described or illustrated, unless expressly prohibited by this disclosure or the nature of the embodiments.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the products, systems, and methods are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A stent delivery system comprising:
a covered stent that is radially expandable from a compressed state to an expanded state and comprises:
a frame configured to urge the covered stent toward the expanded state when the stent is in the compressed state;
a gas-impermeable membrane coupled to the frame; and
a lumen surrounded by the gas-impermeable membrane;
wherein a maximum transverse dimension of the lumen, measured perpendicularly to a longitudinal axis extending between proximal and distal ends of the covered stent, is larger when the covered stent is in the expanded state than when the covered stent is in the compressed state; and
a delivery assistance tube that comprises:
a distal portion comprising:
a distal end of the delivery assistance tube; and
a plurality of openings that extend through an outer wall of the delivery assistance tube;
wherein the distal portion of the delivery assistance tube is configured to be positioned within the lumen of the covered stent; and
a lumen that extends from a proximal end of the delivery assistance tube to the distal portion of the delivery assistance tube and is in fluid communication with the openings.

2. The stent delivery system of claim 1, wherein the openings of the distal portion of the delivery assistance tube comprise a plurality of slits that each extend in a circumferential direction that is substantially perpendicular to a longitudinal axis that extends between the proximal and distal ends of the delivery assistance tube.

3. The stent delivery system of claim 2, wherein:
in the distal portion of the delivery assistance tube, the outer wall of the delivery assistance tube includes:
a plurality of circumferential segments that each extend in the circumferential direction; and
a plurality of axial segments that each extend between two of the circumferential segments in an axial direction that is substantially parallel to the longitudinal axis that extends between the proximal and distal ends of the delivery assistance tube, the axial segments including a plurality of first axial segments, a plurality of second axial segments, a plurality of third axial segments, and a plurality of fourth axial segments;
wherein an angular separation, taken about the longitudinal axis that extends between the proximal and distal ends of the delivery assistance tube:
between each of the first axial segments and each of the second axial segments is approximately 180°;
between each of the third axial segments and each of the fourth axial segments is approximately 180°; and
between each of the first axial segments and each of the third and fourth axial segments is approximately 90°; and
the slits include a plurality of first pairs of slits and a plurality of second pairs of slits, wherein:
for each of the first pairs of slits, two of the circumferential segments, one of the first axial segments, and one of the second axial segments circumscribe each of the slits of the first pair;
for each of the second pairs of slits, two of circumferential segments, one of the third axial segments, and one of the fourth axial segments circumscribe each of the slits of the second pair; and
taken in the axial direction, each of the first pairs of slits is adjacent to at least one of the second pairs of slits.

4. The stent delivery system of claim 3, wherein a collective area of the openings is at least 40% of a surface area of an inner surface of the gas-impermeable membrane.

5. The stent delivery system of claim 4, wherein the distal end of the delivery assistance tube is closed.

6. The stent delivery system of claim 4, wherein:
the lumen of the delivery assistance tube is a first lumen of the delivery assistance tube;
the delivery assistance tube comprises a second lumen that extends from the proximal end of the delivery assistance tube to the distal end of the delivery assistance tube such that the distal end of the delivery assistance tube includes an opening; and
the first lumen surrounds the second lumen.

7. The stent delivery system of claim 6, comprising:
an aspiration tube coupled to the proximal end of the covered stent;
wherein the delivery assistance tube is configured to move axially within a lumen of the aspiration tube.

8. The stent delivery system of claim 7, wherein the delivery assistance tube comprises nitinol.

9. The stent delivery system of claim 8, wherein:
the distal portion of the delivery assistance tube is positioned in the lumen of the covered stent; and
the proximal end of the delivery assistance tube is coupled to a negative pressure source such that the negative pressure source is in fluid communication with the lumen of the delivery assistance tube.

10. The stent delivery system of claim 9, wherein the frame is configured to urge the covered stent toward the expanded state with a force at which, when the covered stent is in the compressed state and a surface surrounds the covered stent, a pressure that the covered stent exerts on the surface is at least 85 kilopascals (kPa).

11. A method for use in thrombus removal, the method comprising:
advancing a first guide tube through vasculature of a patient;
reducing pressure within a lumen of a covered stent that is radially expandable from a compressed state to an expanded state and comprises:
a frame configured to urge the covered stent toward the expanded state when the stent is in the compressed state; and
a gas-impermeable membrane that is coupled to the frame and surrounds the lumen;
wherein a maximum transverse dimension of the lumen, measured perpendicularly to a longitudinal axis extending between proximal and distal ends of the covered stent, is larger when the covered stent is in the expanded state than when the covered stent is in the compressed state;
while pressure within the lumen of the covered stent is reduced and while the covered stent is in the compressed state, advancing the covered stent through the first guide tube;
after advancing the covered stent through the first guide tube, expanding the covered stent from the compressed state to the expanded state in the vasculature of the patient at least by:
positioning the covered stent relative to the first guide tube such that at least a portion of the covered stent is disposed distally of a distal end of the first guide tube; and
increasing pressure within the lumen of the covered stent.

12. The method of claim 11, wherein reducing pressure within the lumen of the covered stent is performed such that the pressure within the lumen decreases by at least 90 kilopascals (kPa).

13. The method of claim 12, wherein increasing pressure within the lumen of the covered stent comprises introducing a liquid into the lumen of the covered stent.

14. The method of claim 13, comprising:
advancing a second guide tube through the vasculature of the patient such that a distal end of the second guide tube is positioned proximally of an internal carotid artery and/or a middle cerebral artery of the patient;
wherein advancing the first guide tube through the vasculature of the patient comprises advancing the first guide tube through the second guide tube and beyond the distal end of the second guide tube such that the distal end of the first guide tube is positioned in the internal carotid artery or the middle cerebral artery.

15. The method of claim 14, comprising aspirating a thrombus into the distal end of the covered stent and through the lumen of the covered stent while the covered stent is in the expanded state.

16. The method of claim 15, wherein reducing pressure within the lumen of the covered stent comprises:
positioning a distal portion of a delivery assistance tube within the lumen of the covered stent, the distal portion comprising:
a distal end of the delivery assistance tube; and
a plurality of openings that extend through an outer wall of the delivery assistance tube; and
reducing pressure at a proximal end of the delivery assistance tube such that gas flows from the distal portion of the delivery assistance tube to the proximal end of the delivery assistance tube through a lumen of the delivery assistance tube.

17. The method of claim 16, wherein the openings of the distal portion of the delivery assistance tube comprise a plurality of slits that each extend in a circumferential direction that is substantially perpendicular to a longitudinal axis that extends between the proximal and distal ends of the delivery assistance tube.

18. The method of claim 17, wherein:
in the distal portion of the delivery assistance tube, the outer wall of the delivery assistance tube includes:
a plurality of circumferential segments that each extend in the circumferential direction; and
a plurality of axial segments that each extend between two of the circumferential segments in an axial direction that is substantially parallel to the longitudinal axis that extends between the proximal and distal ends of the delivery assistance tube, the axial segments including a plurality of first axial segments, a plurality of second axial segments, a plurality of third axial segments, and a plurality of fourth axial segments;
wherein an angular separation, taken about the longitudinal axis that extends between the proximal and distal ends of the delivery assistance tube:
between each of the first axial segments and each of the second axial segments is approximately 180°;
between each of the third axial segments and each of the fourth axial segments is approximately 180°; and
between each of the first axial segments and each of the third and fourth axial segments is approximately 90°; and
the slits include a plurality of first pairs of slits and a plurality of second pairs of slits, wherein:
for each of the first pairs of slits, two of the circumferential segments, one of the first axial segments, and one of the second axial segments circumscribe each of the slits of the first pair;
for each of the second pairs of slits, two of circumferential segments, one of the third axial segments, and one of the fourth axial segments circumscribe each of the slits of the second pair; and
taken in the axial direction, each of the first pairs of slits is adjacent to at least one of the second pairs of slits.

19. The method of claim 18, wherein a collective area of the openings is at least 40% of a surface area of an inner surface of the gas-impermeable membrane.

20. The method of claim 19, wherein the delivery assistance tube comprises nitinol.

21. A stent delivery system comprising:
a covered stent that is radially expandable from a compressed state to an expanded state and comprises:
a frame configured to urge the covered stent toward the expanded state when the stent is in the compressed state;
a gas-impermeable membrane coupled to the frame; and
a lumen surrounded by the gas-impermeable membrane;
wherein a maximum transverse dimension of the lumen, measured perpendicularly to a longitudinal axis extending between proximal and distal ends of the covered stent, is larger when the covered stent is in the expanded state than when the covered stent is in the compressed state;
a pusher configured to be received in the lumen of the covered stent; and
a compliant bead that is coupled to the pusher and configured to sealingly engage an inner surface of a distal portion of the covered stent when the covered stent is in the compressed state such that gas is not permitted to flow from the distal end of the covered stent to a portion of the lumen of the covered stent that is proximal of the compliant bead.

22. The stent delivery system of claim 21, comprising:
an aspiration tube coupled to the proximal end of the covered stent;
wherein the pusher is configured to move axially within a lumen of the aspiration tube.

23. The stent delivery system of claim 22, wherein a volume of the lumen of the aspiration tube is less than or equal to 2 cubic centimeters (cm³).

24. The stent delivery system of claim 23, wherein a maximum external transverse dimension of the aspiration tube, measured perpendicularly to a longitudinal axis extending between proximal and distal ends of the tube, is less than or equal to 1.6 millimeters (mm).

25. The stent delivery system of claim 24, wherein:
the covered stent is in the compressed state;
the pusher is disposed in the lumen of the aspiration tube and the lumen of the covered stent;
the compliant bead is sealingly engaged with the inner surface of the distal portion of the covered stent; and
a proximal end of the aspiration tube is coupled to a negative pressure source such that the negative pressure source is in fluid communication with the lumen of the aspiration tube.

26. The stent delivery system of claim 25, wherein the frame is configured to urge the covered stent toward the expanded state with a force at which, when the covered stent is in the compressed state and a surface surrounds the covered stent, a pressure the covered stent exerts on the surface is at least 85 kilopascals (kPa).

27. The stent delivery system of claim 26, comprising:
a guide tube;
wherein the covered stent is configured to move axially within a lumen of the guide tube.

28. The stent delivery system of claim 27, wherein a maximum transverse dimension of the guide tube, measured perpendicularly to a longitudinal axis that extends between proximal and distal ends of the guide tube, is less than or equal to 2.2 millimeters (mm).

29. The stent delivery system of claim 28, wherein the frame comprises nitinol.
